(19) United States
(12) Patent Application Publication
(10) Pub. No.: US 2025/0058090 A1
(43) Pub. Date: Feb. 20, 2025

(54) Luminal Tissue Isolation Device and Methods for Using Same

(71) Applicant: The Regents of the University of California, Oakland, CA (US)

(72) Inventor: Craig Munroe, San Francisco, CA (US)

(21) Appl. No.: 18/723,599

(22) PCT Filed: Jan. 5, 2023
(86) PCT No.: PCT/US2023/010235

§ 371 Date: Jun. 24, 2024

Related U.S. Application Data
(60) Provisional application No. 63/296,574, filed on Jan. 5, 2022.

(51) Int. Cl. A61M 25/10; A61M 25/00; A61M 25/01

(52) U.S. Cl. CPC A61M 25/10; A61M 25/0108; A61M 2025/0001; A61M 2025/0039

(57) ABSTRACT

Devices and methods for isolating segments of luminal tissue are provided. Aspects of the present invention include catheter devices comprising (a) an outer catheter comprising an outer catheter lumen, (b) an inner catheter positioned within the outer catheter lumen, the inner catheter comprising: (i) first and second inner catheter lumens, (ii) perforations at a distal region of the inner catheter, and (ill) a guidewire passage channel positioned distal to the perforations, (c) a balloon attached to the inner catheter positioned distal to the perforations and proximal to the guidewire passage channel in fluidic communication with the second inner catheter lumen and configured to radially expand, (d) a guidewire positioned in the guidewire passage channel, (e) a first radiopaque marker on the inner catheter distal to the balloon, and (f) a second radiopaque marker at a distal region of the outer catheter.

### CROSS-REFERENCE

This application claims the benefit of U.S. Provisional Patent Application No. 63/296,574, filed Jan. 5, 2021, which applications is incorporated herein by reference in its entirety.

### INTRODUCTION

The gastrointestinal tract can be obstructed for reasons such as cancer, benign strictures due to peptic ulcer disease, inflammatory bowel disease and other etiologies. These obstructions can result in severe symptoms including pain, nausea, vomiting and weight loss. When cancer is the cause of the obstruction, malignant gastric outlet obstruction (MGOO) is associated with a 7-20 week mean survival time after diagnosis

Intraluminal stents can be used to allow passage of solids and liquids past an obstruction. However, stents often work poorly due to occlusion with food, restenosis with cancer, overgrowth with hyperplastic tissue, incomplete relief of stricture, pain, infection, migration, bleeding, perforation, fistula formation and stent mis-deployment.

Clinicians and patients would benefit from devices that facilitate the bypassing of the obstruction completely endoscopically. These bypass procedures are now commonly performed at specialty medical centers by creation of endoscopic gastrojejunostomy in the case of benign and malignant gastric outlet obstruction. Unfortunately, this procedure is very challenging and widespread adoption is limited due to challenges in identifying and isolating a suitable segment of intestine. For example, in some cases, adoption is limited due to challenges identifying segments of luminal tissue in order to safely deploy a lumen apposing metal stent, or other devices designed for lumen apposition.

### SUMMARY

Thus, there is a need for improved and useful devices and methods for isolating segments of luminal tissue, such as gastrointestinal tissue, including jejunal tissue, that can be used to facilitate rapid and accurate isolation of segments of the gastrointestinal tract for safe and effective therapeutic interventional endoscopy. Embodiments of the invention described herein provide such new and useful devices and methods. Such devices and methods will aid in facilitating a higher quality of life, longer stent patency and safer treatment options for clinicians compared to self-expanding metal stent (SEMS) procedures and surgery currently applied, by simplifying the procedure and thus making it more available to patients throughout the world.

Embodiments of devices and methods of the present invention can be used to isolate segments of luminal tissue by pressurizing segments of luminal tissue, such as the bowel, with turbulent liquid, including under endoscopic ultrasonographic guidance and with the assistance of X-ray fluoroscopy, to allow the creation an ostomy or fistula without the need for surgical intervention. The invention is used to isolate segments of the luminal gastrointestinal tract that have been affected by complete or partial obstruction, such as, for example, resulting from MGOO. Embodiments of devices according to the present invention consist of endoscopic catheters that can be advanced over wire guidance past the level of luminal obstruction. After advancing an inner catheter and an insufflating, donut-shaped distal balloon, the catheter device can be instilled with a mixture of water and contrast and the balloon filled to isolate a segment of bowel. The device handle can be fixed or removable, and under 3.7 mm in diameter for example, to allow passage through the working channel of a therapeutic upper endoscope for rapid exchange.

Devices and methods for isolating segments of luminal tissue are provided. Aspects of the present invention include catheter devices comprising (a) an outer catheter comprising an outer catheter lumen, (b) an inner catheter positioned within the outer catheter lumen, extending beyond the distal end of the outer catheter, the inner catheter comprising: (i) first and second inner catheter lumens, (ii) perforations at a distal region of the inner catheter configured to allow fluid to exit the first inner catheter lumen in a turbulent flow, and (iii) a guidewire passage channel positioned distal to the perforations, wherein the inner catheter is configured to move laterally within the outer catheter lumen, (c) a balloon attached to the inner catheter positioned distal to the perforations and proximal to the guidewire passage channel in fluidic communication with the second inner catheter lumen and configured to radially expand, (d) a guidewire positioned in the guidewire passage channel, (e) a first radiopaque marker on the inner catheter distal to the balloon, and (f) a second radiopaque marker at a distal region of the outer catheter. Also provided are methods for isolating a segment of luminal tissue.

The devices and methods find use in a variety of different applications, e.g., the treatment of subjects with complete or partial obstruction of the gastrointestinal tract, such as, for example, subjects with luminal tissue affected by MGOO.

### BRIEF DESCRIPTION OF THE FIGURES

The invention may be best understood from the following detailed description when read in conjunction with the accompanying drawings. Included in the drawings are the following figures:

FIG. 1 depicts an embodiment of the device according to the present disclosure.

FIG. 2 depicts a side view of a distal region of an embodiment of a device according to the present disclosure.

FIG. 3 depicts a cross sectional view of a distal region of an embodiment of a device according to the present disclosure.

FIGS. 4A-4D depict views of a distal region of an embodiment of a device according to the present disclosure.

FIG. 5 depicts a cross sectional view of an alternative embodiment of a device according to the present disclosure.

FIG. 6 depicts a cutaway view of an endoscopic ultrasound guided gastroenterostomy procedure in luminal tissue of a subject.

FIG. 7 depicts an example of a lumen apposing metal stent.

FIGS. 8A-C depict views of a benchtop testing environment.

FIGS. 9A-D depict views of an in vivo testing environment as well as imaging results of such in vivo testing.

### DETAILED DESCRIPTION

Aspects of the present invention include catheter devices comprising (a) an outer catheter comprising an outer catheter lumen, (b) an inner catheter positioned within the outer catheter lumen, extending beyond the distal end of the outer catheter, the inner catheter comprising: (i) first and second inner catheter lumens, (ii) perforations at a distal region of the inner catheter configured to allow fluid to exit the first inner catheter lumen in a turbulent flow, and (iii) a guidewire passage channel positioned distal to the perforations, wherein the inner catheter is configured to move laterally within the outer catheter lumen, (c) a balloon attached to the inner catheter positioned distal to the perforations and proximal to the guidewire passage channel in fluidic communication with the second inner catheter lumen and configured to radially expand, (d) a guidewire positioned in the guidewire passage channel, (e) a first radiopaque marker on the inner catheter distal to the balloon, and (f) a second radiopaque marker at a distal region of the outer catheter. Also provided are methods for isolating a segment of luminal tissue.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the device and method may be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, the present disclosure provides devices and methods for isolating a segment of luminal tissue. By isolating a segment of luminal tissue, it is meant pressurizing a segment of luminal tissue by expanding a balloon catheter to engage with the luminal wall while also ejecting liquid from the device in a turbulent flow proximal of the balloon where to facilitate visualizing the segment of luminal tissue using ultrasound sonography and/or X-ray fluoroscopy. Pressurizing a segment of luminal tissue as such facilitates identifying the segment of luminal tissue, for example, under ultrasound sonography, and accessing the segment for purposes of creating an ostomy or fistula in the luminal tissue. In general, the luminal tissue is luminal tissue of a human subject that may be male or female and of any body type or composition.

### Devices for Isolating Segments of Luminal Tissue

Aspects of the present disclosure include devices for isolating segments of luminal tissue. In particular, the present disclosure includes catheter devices for isolating segments of luminal tissue comprising: (a) an outer catheter comprising an outer catheter lumen, (b) an inner catheter positioned within the outer catheter lumen, extending beyond the distal end of the outer catheter, the inner catheter comprising: (i) first and second inner catheter lumens, (ii) perforations at a distal region of the inner catheter configured to allow fluid to exit the first inner catheter lumen in a turbulent flow, and (iii) a guidewire passage channel positioned distal to the perforations, wherein the inner catheter is configured to move laterally within the outer catheter lumen, (c) a balloon attached to the inner catheter positioned distal to the perforations and proximal to the guidewire passage channel in fluidic communication with the second inner catheter lumen and configured to radially expand, (d) a guidewire positioned in the guidewire passage channel, (e) a first radiopaque marker on the inner catheter distal to the balloon, and (f) a second radiopaque marker at a distal region of the outer catheter. In certain embodiments, the distal end of the device may further include a sensor, such as radiowave sensor, for determining position of the distal end of the device in vivo.

### Outer Catheter:

Catheter devices of the invention comprise an outer catheter. The outer catheter comprises a catheter with a hollow lumen, referred to as an outer catheter lumen. In embodiments, the outer catheter is made of any convenient biocompatible material. The outer catheter may be configured so as to provide support for an inner catheter positioned within the outer catheter lumen, and, in particular, the outer catheter may be configured to provide support for a distal region of the inner catheter that extends beyond the distal end of the outer catheter. In embodiments, such support may be provided by the material used to make the outer catheter or other design characteristics of the outer catheter, such as the thickness of the outer catheter wall. In some cases, the outer catheter is made of a polymeric material, such as, for example, a polymer conventionally used in biliary stents or polytetrafluoroethylene (PTFE) or the like. In embodiments, the outer catheter may comprise any convenient outer diameter, such as an outer diameter between approximately 3 Fr and 34 Fr, such as 10 Fr. In embodiments, the outer catheter may comprise any convenient lateral length, such as a length between approximately 100 cm and 800 cm, such as between 100 cm and 300 cm or 200 cm and 400 cm or 400 cm and 600 cm or 600 cm and 800 cm. The outer catheter lumen comprises a hollow cylindrical space extending over the length of the outer catheter and configured to fit and hold the inner catheter positioned within the outer catheter lumen. In embodiments, any convenient wall thickness of the outer catheter may be used and may vary, for example, based on aspects of the inner catheter, such as the diameter or the size of the balloon present on the inner catheter. In embodiments, the outer catheter lumen may comprise any convenient diameter (i.e., the inner diameter of the outer catheter) configured to hold the inner catheter within the outer catheter lumen and can range between 3 Fr and 34 Fr, such as 5 Fr, 6 Fr, 7 Fr, 8 Fr or 9 Fr. In embodiments, the outer catheter lumen is configured to accommodate a 7 Fr inner catheter present in the outer catheter lumen.

### Inner Catheter

Catheter devices of the invention further comprise an inner catheter. In embodiments, the inner catheter may be configured to extend beyond the distal end of outer catheter between 0 and 100 cm, such as between 5 and 20 cm, and such length may vary. In embodiments, the inner catheter is configured to move laterally within outer catheter lumen of outer catheter. That is, inner catheter can be further extended beyond the distal end of outer catheter, such as during application of device, i.e., while the catheter device is positioned within a subject during an endoscopic procedure. In embodiments, lateral movement of the inner catheter within the outer catheter lumen comprises extending the distal end of the inner catheter further beyond the distal end of the outer catheter. In embodiments, the inner catheter is configured to move laterally within the outer catheter lumen by 0 to 50 cm, such as 10 cm, 20 cm, 30 cm, 40 cm or 50 cm. In embodiments, lateral movement of the inner catheter is controlled by manipulating the inner catheter at a proximal region of the device.

In embodiments, the inner catheter may be made of any convenient biocompatible material. For example, embodiments of the inner catheter may be made of a material that enables the inner catheter to extend beyond the distal end of the outer catheter while maintaining structural integrity, i.e., without folding, unexpectedly bending, collapsing or otherwise becoming unintentionally misaligned. In some cases, the inner catheter comprises a polymeric material, such as, for example, a polymer conventionally used in biliary stents or polytetrafluoroethylene (PTFE) or the like. In embodiments, the inner catheter may comprise any convenient outer diameter, such as an outer diameter between approximately 3 Fr and 34 Fr, such as 7 Fr. Inner catheter may comprise any convenient lateral length, such as a length between approximately 100 cm and 800 cm, such as between 100 cm and 300 cm or 200 cm and 400 cm or 400 cm and 600 cm or 600 cm and 800 cm. In general, in embodiments, the length of the inner catheter is substantially similar to, and greater than, the length of the outer catheter.

In certain embodiments, the inner catheter may be self-propelling. By “self-propelling,” it is meant that the device may be configured so that the inner catheter automatically advances further along the guidewire in a distal direction in certain circumstances. For example, the device may be configured so that the inner catheter automatically advances in a distal direction relative to the distal end of the outer catheter. Any convenient means may be used to advance the inner catheter to make it self-propelling. For example, the inner catheter may be operably connected to a pressure source that applies pressure to, for example, a proximal end of the inner catheter, or the proximal end of the inner catheter may be operably connected to a mechanical translation system, such as a spring or a motor or a ratchet system or the like, in each case configured to advance the position of the inner catheter in a distal direction. In some cases, the device may be configured so that pressure applied to fluid in either or both of the first and second inner catheter lumens is applied to propel the inner catheter in a further distal direction. In some cases, the device may be configured so that prior to filling the balloon, the inner catheter is automatically further advanced in a distal direction.

### Inner Catheter Lumens

Catheter devices of the invention further comprise inner catheter lumens. In embodiments, the inner catheter comprises first and second inner catheter lumens extending all, or substantially all, the lateral length of inner catheter. First and second inner catheter lumens are fluidically isolated from each other so that they can be separately filled with different fluids and separately pressurized to different pressures. Fluid and/or pressure may be applied to first and second inner catheter lumens by applying fluid and/or pressure sources at the proximal ends of first and second inner catheter lumens.

In embodiments, the first and second inner catheter lumens of the inner catheter extend the length of the inner catheter and are configured to separately and independently receive fluid and withstand pressurization. In some cases, the first and second inner catheter lumens are positioned side by side. In other cases, the first and second inner catheter lumens are positioned in a coaxial configuration. In some cases, the proximal ends of the first and second inner catheters are configured to be attached to pressure and/or fluid sources. For example, in embodiments, the proximal ends of the first and/or second inner catheters comprise fixed or removeable Luer locks.

In other embodiments, the inner catheter comprises a third inner catheter lumen. In some cases, the third inner catheter lumen is configured to receive a guidewire.

### Fluids in Inner Catheter Lumens:

In embodiments, catheter devices may further comprise fluids present in the first and second inner catheter lumens. In embodiments, fluid present in the first inner catheter lumen comprises a first contrast. In such embodiments, fluid present in the first inner catheter lumen may further comprise water. In other embodiments, fluid present in the second inner catheter lumen comprises a second contrast. In such embodiments, fluid present in the first inner catheter lumen may further comprise a saline solution.

### Perforations:

Catheter devices of the invention further comprise perforations. In embodiments, present at a distal region of the inner catheter are perforations. In embodiments, the perforations are configured to fluidically connect the first inner catheter lumen with space exterior to the inner catheter. i.e., the luminal tissue in which a distal region of the embodiment of the catheter device is positioned during use, such as during endoscopic procedures. The perforations are further configured to allow fluid to exit the first inner catheter lumen in a turbulent flow. By “turbulent flow,” it is meant an agitated flow sufficient that the turbulence can be visualized using, for example, ultrasound imaging technology. Catheter devices according to the invention may be configured so that the turbulent flow can be used to identify, isolate or visualize a segment of the luminal tissue proximal to such turbulent flow. In some cases, the catheter device is configured to pressurize segments of the luminal tissue using the turbulent flow of fluid exiting the first inner catheter lumen of inner catheter lumen. In other cases, the catheter device is configured so that the turbulent flow of fluid exiting the first inner catheter lumen identifies a distal segment of luminal tissue for creation of an ostomy.

In embodiments, the perforations may be located on the inner catheter at a distal region and may be shaped in such a manner to maximize turbulent flow upon pressurization of fluid present the first inner lumen of the inner catheter. Any convenient number of perforations may be present on the inner catheter, such as between 1 and 1,000 or more, such as, for example, 10, 20, 50, 100 or 500 perforations. In embodiments, the perforations may be round, though that need not always be the case, and may have any convenient diameter, such as diameters between 0.001 mm and 1 mm or more, such as 0.05 mm or 0.1 mm or 0.5 mm. In embodiments, the perforations are present in a distal region of the inner catheter capable of extending beyond the distal end of the outer catheter. That is, in embodiments, the outer catheter, the inner catheter and the perforations are configured so that the inner catheter can be extended beyond the distal end of the outer catheter such that all the perforations are exposed to the luminal tissue, i.e., not covered by the outer catheter.

### Guidewire and Guidewire Passage Channel:

Catheter devices of the invention further comprise a guidewire passage channel and a guidewire. In embodiments, present on the inner catheter at a distal region of the inner catheter is a guidewire passage channel. In embodiments, the guidewire passage channel is positioned distal to the perforations near the distal end of the inner catheter. In embodiments, the guidewire passage channel is configured to enclose a region of a guidewire (i.e., the guidewire is threaded through the guidewire passage channel), such that the catheter device can be positioned in the luminal tissue via guidewire guidance (i.e., over wire guidance). In some cases, the guidewire passage channel comprises a ring or ring-like structure configured to enclose a region of a guidewire (i.e., to allow the guidewire to be threaded through the guidewire passage channel). In other cases, the guidewire passage channel comprises a tube or tube-like structure extending along a distal region of the inner catheter. In embodiments, the guidewire passage channel may made from any convenient biocompatible material, such as the same material or different from that of the inner catheter and may be affixed to the inner catheter by any convenient biocompatible bonding or gluing process, such as, for example, application of an epoxy or an adhesive.

In embodiments, the guidewire may be any convenient guidewire capable of providing guidance of the distal end of the catheter device into the desired location of luminal tissue and anatomy in a subject. In some cases, the guidewire may be a standard, i.e., off the shelf, endoscopic retrograde cholangiopancreatography (ERCP). In some cases, the guidewire may be a wire with a diameter of 0.035″ or smaller. In embodiments, the guidewire and the guidewire passage channel are configured to direct distal regions of the inner catheter and the outer catheter to a desired position in the luminal tissue or anatomy. In embodiments of the catheter device, the device is configured so that the guidewire remains in a fixed position relative to the luminal tissue and the inner catheter and the outer catheter move laterally within the luminal tissue along the guidewire. In some cases, the device is configured to be advanced over the guidewire past a luminal obstruction, such as, for example, a malignant gastric outlet obstruction (MGOO).

### Balloon:

Catheter devices of the invention further comprise a balloon. In embodiments of the catheter device, the device further comprises a balloon attached to the inner catheter at a distal region of the inner catheter that extends beyond the distal end of outer catheter. In embodiments, the balloon is located at a position distal to the perforations and proximal to the guidewire passage channel. The balloon is configured so that the interior of the balloon is in fluidic communication with the second inner catheter lumen. That is, fluid present in the second inner catheter lumen can be induced to flow into the balloon upon application of pressure, such as via a pressure source, to the second inner catheter lumen. In embodiments, the balloon is configured to radially expand. That is, in embodiments, upon application of pressure to fluid present in the second inner catheter lumen, fluid is induced to flow into the balloon causing the balloon to expand radially. In embodiments, the balloon is configured to be inflated using fluid present in the second inner catheter lumen only (as distinct from fluid present in the first inner catheter lumen). In embodiments, the balloon is configured to expand upon inflation such that the exterior surface of the balloon engages with the luminal tissue in which the device is positioned. In embodiments, the balloon is configured to engage the luminal tissue to occlude a section of the luminal tissue upon inflation. That is, in some cases, the balloon is configured to engage with the luminal tissue in order that turbulent flow of fluid ejected from the perforations pressurizes a region of the luminal tissue proximal to the balloon. Such a pressurized segment of luminal tissue facilitates isolating and visualizing the segment of luminal tissue, for example, in connection with performing an endoscopic procedure. In embodiments, the balloon may be configured in any convenient shape to facilitate engaging the luminal tissue upon inflation. In some embodiments, the balloon is configured such that the balloon is donut shaped or torus shaped upon inflation. In embodiments, the balloon may be made from any convenient biocompatible material. For example, in embodiments, the balloon is made from a polymeric material, such as an expandable polymer, including in some cases, expandable polymers used for standard endoscopic retrograde cholangiopancreatography (ERCP) balloons.

### Pressure Sources:

Embodiments of the invention may further comprise a first pressure source configured inflate the balloon. In embodiments, the first pressure source is in fluidic communication with the first inner catheter lumen and the balloon. In some cases, the first pressure source is a syringe, such as, for example, a pre-filled syringe.

Embodiments of the invention may further comprise a second pressure source configured to cause fluid to exit the first inner catheter lumen through the perforations in a turbulent flow. In embodiments, the second pressure source is in fluidic communication with the second inner catheter lumen. In some cases, the second pressure source is a syringe, such as, for example, a pre-filled syringe.

### Pressure Sensors:

Embodiments of the invention may further comprise a pressure sensor configured to measure pressure inside the balloon. In such embodiments, the pressure sensor may be positioned on the surface of the balloon. In other cases, the pressure sensor may be located inside the balloon. In embodiments, the pressure sensor may be configured to wirelessly communicate with a monitor positioned outside the luminal tissue.

In some embodiments, the device may be configured to automatically inflate the balloon to a specified pressure. For example, in embodiments, the device further comprises a pressure source attached to the second inner catheter lumen, and a pressure sensor attached to the balloon, wherein the pressure source and pressure sensor are configured to automatically inflate the balloon to a specified pressure.

### Radiopaque Markers:

Catheter devices of the invention further comprise a first radiopaque marker on the inner catheter distal to the balloon and a second radiopaque marker at a distal region of the outer catheter. In embodiments, the first and second radiopaque markers are any convenient marker configured for visualization using fluoroscopy, in particular, X-ray fluoroscopy. That is, the first and second radiopaque markers are opaque with respect to fluoroscopy, such as X-ray imaging. In embodiments, the first and second radiopaque markers are positioned on the catheter device such that visualizing the markers facilitates visualizing the location and/or configuration of the device within the luminal tissue. In some cases, visualizing the first and second radiopaque markers facilitates visualizing the position of the inner catheter with respect to the outer catheter and further facilitates locating, identifying and isolating a segment of luminal tissue, such as a segment of the luminal tissue subject to an endoscopic procedure. In some cases, visualizing the first and second radiopaque markers enables measuring how far the distal end of the inner catheter extends beyond the distal end of the outer catheter. Such measurements may be used to identify an isolated segment of luminal tissue.

In embodiments, the first radiopaque marker is present on the inner catheter in a distal region that is distal to the balloon and proximal to the guidewire passage channel. In embodiments, the second radiopaque marker is present on the outer catheter in a distal region near the distal end of the outer catheter. In embodiments, the first and second radiopaque markers may be made from any convenient biocompatible material that is opaque with respect to fluoroscopy, i.e., opaque with respect to X-rays, such as a metal such as lead or a material comprising lead. In embodiments, the first and second radiopaque markers may include distinct features visible via fluoroscopy such that they can be distinguished from each other when fluoroscopically imaged. In embodiments, the first and second radiopaque markers may be affixed to inner and outer catheters, respectively, via any convenient biocompatible bonding or gluing process, such as, for example, application of an epoxy or an adhesive. In some cases, the first and/or second radiopaque markers are crimped or pressed or press fit onto the inner and/or outer catheters, respectively.

In some cases, embodiments of the device comprise additional radiopaque markers. Such additional markers may be present on, for example, the inner catheter or the outer catheter or the balloon or the guidewire. In embodiments, the radiopaque markers are configured to mark specified distances on the inner catheter. That is, a plurality of radiopaque markers may be spaced a series of fixed distances along a distal region of the inner catheter.

In embodiments, one or more of the radiopaque markers comprises a magnet. Such an embodiment enables a section of the catheter device (the section to which the magnetic radiopaque marker is attached) to interact with other instruments, such as other instruments used in an endoscopic procedure, that similarly comprise a magnet. For example, in some cases, a magnet on the first or second radiopaque marker is configured to oppose a second magnet present on another device, such as a device present in another location within the luminal tissue or elsewhere, such as, for example, an anastomosis device. Such configuration may allow, for example, the magnet of the first radiopaque marker and the second magnet attached to another device to draw the luminal tissue proximal to the first radiopaque marker closer to tissue where the second magnet on the other device, such as an anastomosis device, is present. That is, the first radiopaque marker and the second magnet attached to another device may, through the force of the two magnets attracting each other, draw a segment of luminal tissue that has been identified, visualized and/or isolated using an embodiment of the device towards another instrument present in other tissue, such as, for example, a stomach of a subject or another segment of luminal tissue. In some cases, the magnet on first radiopaque marker and the second magnet present on another device are configured to draw jejunal tissue closer to stomach tissue. Such interaction between tissues caused by one or more magnets attached to embodiments of the device may facilitate various endoscopic procedures, such as, for example, using an embodiment of the device in connection with an endoscopic gastrojejunostomy procedure.

### Locking Mechanism:

Embodiments of the catheter device may further comprise a locking mechanism present at a proximal region of the outer catheter. In embodiments, the locking mechanism is configured to fix a length at which the inner catheter extends beyond the distal end of the outer catheter. When using an embodiment of the catheter device, the inner catheter may be moved laterally relative to the outer catheter, such that the distal end of the inner catheter extends beyond the distal end of the outer catheter at different lengths. During certain aspects of procedures in which an embodiment of the catheter device is employed, the inner catheter may be substantially retracted within the outer catheter, and, during other aspects of such procedures, the inner catheter may be extended further, laterally, within the outer catheter, such that the balloon and the perforations extend further beyond the distal end of the outer catheter. Extending the distal end of the inner catheter may facilitate using embodiments of the catheter device to access luminal tissue, or other parts of a subject's anatomy, that would otherwise not be accessible, due to for example, the small diameter of such luminal tissue or forces applied to the device, in particular at or near the distal end of the catheter device when maneuvering the device in luminal tissue.

By “fix a length,” it is meant that the locking mechanism is configured to hold the lateral position of the inner catheter relative to the outer catheter constant so that the inner catheter, for example, can no longer extend further beyond the distal end of the outer catheter. In embodiments, the locking mechanism may be present on the outer catheter and may function by any convenient mechanism. For example, in embodiments, the locking mechanism may work by applying pressure from the outer catheter to the inner catheter, such as via a clamping mechanism, or the locking mechanism may work by applying force to the inner catheter from the outer catheter via a threaded screw-type mechanism.

In embodiments, the locking mechanism may be configured to indicate the length at which the inner catheter extends beyond the distal end of the outer catheter. For example, in embodiments, the locking mechanism may include a dial or gauge or other display configured to indicate a relative, lateral, distance between the distal ends of the inner and outer catheters. Such a gauge or display indicating relative distance between the inner and outer catheters may be configured as a reference mark on the outer catheter with a window that allows markings printed on the inner catheter to be viewed relative to the reference mark on the outer catheter.

As described above, in embodiments, the locking mechanism may be present on the outer catheter and further may be present at a proximal region of the outer catheter. That is, in embodiments, the locking mechanism may be positioned so that during use of the catheter device, the locking mechanism remains external to the luminal tissue, and, in particular, external to the subject, such that the locking mechanism is viewable and accessible to an operator of the device during use.

### Other Attachments to Device:

In embodiments, the distal region of the catheter device may further comprise a camera. Any convenient endoscopic camera may be attached to the inner or outer catheters of the catheter device such that luminal tissue of interest, such as luminal tissue that is isolated using the device in connection with an endoscopic procedure, may be further visualized. Cameras of interest may communicate with receivers outside the luminal tissue via a wired or wireless communication protocol.

In other embodiments, the distal region of the catheter device may further comprise a needle. Any convenient needle may be attached to the inner or outer catheters of the catheter device. A needle may be attached in such a configuration that luminal tissue, such as obstructed luminal tissue, may be punctured using the needle. In embodiments that comprise a needle, the device may further comprise suitable controls for use in steering a distal region of the device or exposing the needle or otherwise manipulating the needle vis-à-vis the luminal tissue.

In embodiments, the device further comprises a detachable handle positioned at the proximal end of the device and configured to allow an endoscope to be entered and removed over the outer catheter. Any convenient, off-the-shelf handle for endoscopic catheters may be used and attached to the device for manipulating the device by an operator when the device is applied to luminal tissue of a subject. The handle may be configured to be removable from the device and/or to allow the device to be passed through the handle or otherwise attached to the handle. In some embodiments, the device comprising a handle is configured to allow passage of the handle through a working channel of a therapeutic upper endoscope. In other embodiments, the handle is configured to be under 3.7 mm in diameter.

### Luminal Tissue:

In embodiments, the luminal tissue comprises enteral tissue. In other embodiments the luminal tissue comprises a gastrointestinal lumen. In embodiments, the luminal tissue is luminal tissue of a subject. In some embodiments, the subject is human. The human subject may be male or female and of any body type or composition. While the subject may be of any age, in some instances, the subject is not an adult, such as a toddler, juvenile, child, etc. While the subject may be of any body type or body size, in some instances, the subject does not exhibit normal body mass index, such as an underweight, overweight or obese subject. In other embodiments, the subject may be an animal that is not human (that is, in “non-human subjects”) such as, but not limited to, birds, mice, rats, dogs, cats, livestock and horses.

### Applications

The device may be configured for use in connection with any relevant and applicable endoscopic procedure in which endoscopically isolating luminal tissue is beneficial. In embodiments, the device is configured to treat a subject for Malignant Gastric Outlet Obstruction (MGOO). As described above, Malignant Gastric Outlet Obstruction (MGOO), caused by an intestinal blockage from cancer either inside or outside of the bowel, is associated with an approximately six-month median survival from symptom onset. This condition causes significant distress to patients, with symptoms including nausea, intractable vomiting, dehydration, poor nutrition, and malaise in cancer patients. See, e.g., ASGE Standards of Practice Committee, Jue T L, et al. (ASGE Standards of Practice Committee Chair, 2017-2020); ASGE guideline on the role of endoscopy in the management of benign and malignant gastroduodenal obstruction. Gastrointest Endosc. 2021 February; 93 (2): 309-322.e4. doi: 10.1016/j.gie.2020.07.063; incorporated herein by reference. In the West, MGOO occurs most commonly from obstruction of the duodenum by pancreatic cancer. In 2021, 60,000 people in the US will be diagnosed with pancreatic cancer (˜7% of all cancer deaths), and one quarter of these patients will present with MGOO. MGOO is most commonly from gastric cancer (primary or post-surgical) in Asian countries but can also occur due to a variety of other conditions including metastatic disease, primary small bowel cancer and other malignant and benign conditions such as peptic ulcer disease, scar tissue, SMA syndrome and inflammatory bowel disease.

In embodiments, the device is configured to treat a subject for benign gastric outlet obstruction. In other embodiments, the device is configured to treat a subject affected by complete or partial obstruction of luminal gastrointestinal tract. With respect to specific treatments for conditions, such as those described above, in embodiments, the device is configured to facilitate creation an ostomy or fistula in a subject. In other embodiments, the device is configured to facilitate therapeutic interventional endoscopy in a subject. In some treatments, the device is configured to facilitate placement of a lumen-apposing metal stent (LAMS) in a subject.

In embodiments, the device is configured to facilitate an endoscopic ultrasound guided gastroenterostomy (EUS-GE) procedure in a subject. This outpatient procedure involves placement of a lumen-apposing metal stent (LAMS) under endoscopic ultrasonographic guidance. Although a new technique, case series report a technical success rate of 90%. Importantly, EUS-GE is associated a with lower recurrence of outlet obstruction symptoms and need for reintervention compared to enteral stenting, likely because EUS-GE allows for complete enteral bypass like what is accomplished with surgery. The procedure does not carry the morbidity and mortality associated with surgical bypass (which has a median length of stay of 14-24 days). EUS-GE however is technically very challenging, and widespread adoption into non-academic practices is limited due to difficulties in identifying and isolating a suitable segment of intestine to safely deploy a LAMS or other devices designed for lumen apposition. In trained expert hands, the procedure still carries a >15% risk of serious adverse events. The limitation of jejunal isolation has therefore resulted in the procedure not being widely adopted, despite excellent patient outcomes. See. e.g., Ge P S, et al. EUS-guided gastroenterostomy versus enteral stent placement for palliation of malignant gastric outlet obstruction. Surg Endosc. 2019; 33 (10): 3404-3411 (doi: 10.1007/s00464-018-06636-3); Carbajo A Y, et al. Clinical Review of EUS-guided Gastroenterostomy (EUS-GE). J Clin Gastroenterol. 2020 January; 54 (1): 1-7 (doi: 10.1097/MCG.0000000000001262) (PMID: 31567785); incorporated herein by reference. In other embodiments, the device is configured to facilitate an endoscopic gastrojejunostomy procedure in a subject.

In addition to those configurations described above, embodiments of the device may be configured to facilitate a range of other procedures related to a range of conditions, such as, for example, the device may be configured to facilitate, or may otherwise be configured for use in connection with: percutaneous procedures, such as, for example, percutaneous jejunostomy formation procedures; isolating segments of the bile duct and liver for interventions using percutaneous and endoscopic techniques; treatment of obstructions of the colon and ileocecal valve; intravascular interventions using interventional radiology techniques; combined transhepatic and percutaneous small bowel interventions; percutaneous management of large and small bowel obstruction, or for purposes of feeding and nutrition, via fluoroscopy guided interventions through the entire length of the gastrointestinal tract, using X-ray fluoroscopy or other modalities, including other imaging modalities.

### Exemplary Embodiments

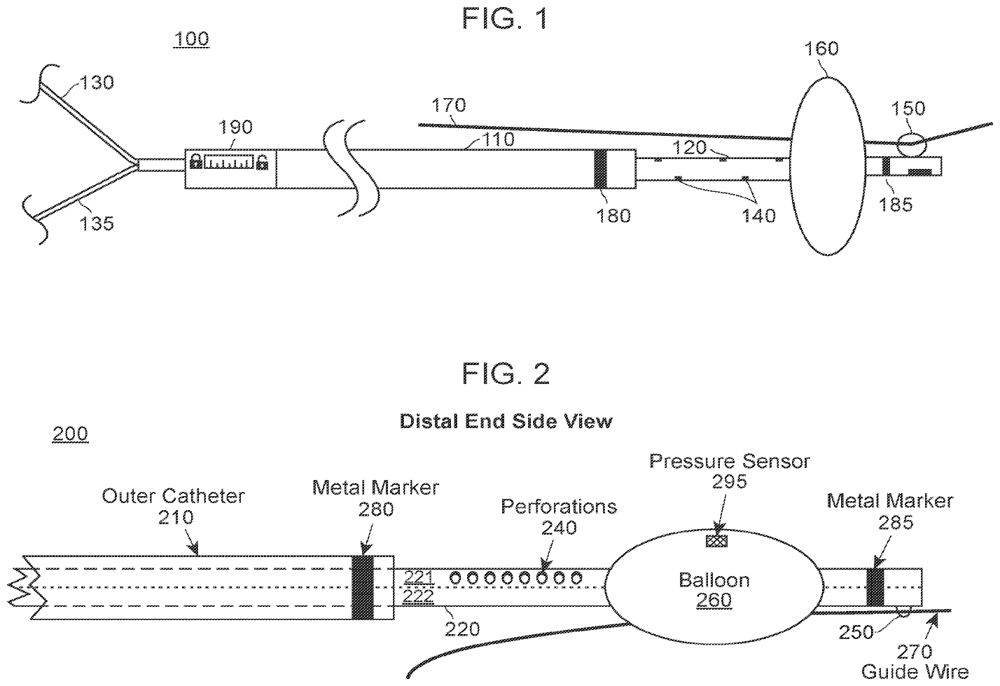

Aspects of the claimed invention are described in connection with embodiments of the device depicted in FIGS. 1-4 for ease of illustration only and without limiting the present invention to such embodiments. FIG. 1 depicts an embodiment of device 100 for isolating luminal tissue according to some aspects of the present disclosure. The right-hand side of FIG. 1 corresponds to a distal region of device 100, and the left-hand side of FIG. 1 corresponds to a proximal region of device 100. The distal region of device 100 is subject-facing when the device is positioned within luminal tissue, such as gastrointestinal tissue including the jejunum. The middle section of device 100 is elided, as depicted by the dividing lines in the middle of FIG. 1, to indicate the diagram is not shown to scale with respect to the lateral length of device 100.

Outer catheter 110 is depicted horizontally in FIG. 1, with a distal region of outer catheter 110 on the right side of the figure and a proximal region of outer catheter 110 on the left side of the figure. Outer catheter 110 comprises an outer catheter lumen, in which inner catheter 120 is positioned. In particular, inner catheter 120 is depicted as being positioned within outer catheter lumen of outer catheter 110 such that a distal region of inner catheter 120 extends beyond the distal end of outer catheter 110.

As described above, inner catheter 120 comprises first and second inner catheters 130, 135. The proximal end of first inner catheter lumen 130 is depicted as extending proximal to the proximal end of outer catheter 110. The proximal end of second inner catheter lumen 135 is depicted as extending proximal to the proximal end of outer catheter 110. First and second inner catheter lumens are separate and distinct such that they may each comprise different fluids and may be pressurized to different pressures.

Present at a distal region of inner catheter 120 that extends beyond the distal end of outer catheter 110 are perforations 140. Perforations 140 are shown as a series of holds in inner catheter 120 exposing first inner catheter lumen 130 to the luminal tissue, such that perforations 140 allow fluid to exit the first inner catheter lumen in a turbulent flow.

Also present on inner catheter 120 distal to perforations 140 is balloon 160. Balloon 160 is depicted in a substantially inflated state with rounded, donut-shaped sides configured to engage the walls of the surrounding luminal tissue. As described above, balloon 160 may be inflated by pressurizing fluid present within second inner catheter lumen 135 such that fluid is displaced from second inner catheter lumen 135 into balloon 160.

Also present on inner catheter 120 distal to perforations 140 and distal to balloon 160 is guidewire passage channel 150. Guidewire passage channel 150 is depicted as a ring attached to a distal region of inner catheter 120, through which guidewire 170 is threaded. A segment of guidewire 170 is depicted at a distal region of device 100 threaded through guidewire passage channel 150.

Also present on device 100 are first and second radiopaque markers 185 and 180. First radiopaque marker 185 is shown attached to inner catheter 120 near the distal end of inner catheter 120. Second radiopaque marker 180 is shown attached to outer catheter 110 near the distal end of outer catheter 110. As described above, radiopaque markers 185, 180 may be visualized via fluoroscopy to identify a location of device 100 in luminal tissue as well as to identify aspects of the luminal tissue isolated using device 100.

Also present on device 100 is locking mechanism 190. As described above, locking mechanism 190 may be used to fix the distance at which the distal end of inner catheter 120 extends beyond the distal end of outer catheter 110. Locking mechanism 190 is present at a proximal region of outer catheter 110 and comprises a dial identifying the relative distance that inner catheter 120 extends beyond outer catheter 110.

FIG. 2 depicts a distal region of an embodiment of device 200 for isolating luminal tissue according to some aspects of the present disclosure, similar to device 100 depicted in FIG. 1. The right-hand side of FIG. 2 corresponds to a distal region of device 200, and the left-hand side of FIG. 2 corresponds to a proximal region of device 200. The distal region of device 200 is subject-facing and when the device is positioned within luminal tissue, such as gastrointestinal tissue including the jejunum. The proximal region of device 200, which is the operator-facing (i.e., away from the subject) region of the device, is not shown in FIG. 2.

Present on device 200, seen on the left side of FIG. 2, is outer catheter 210. For illustrative purposes, outer catheter 210 is depicted as transparent in order to show that outer catheter 210 comprises an outer catheter lumen, in which inner catheter 220 is present. The outer catheter lumen is sized appropriately such that inner catheter 220 fits within the outer catheter lumen, allowing inner catheter 220 to be moved laterally with respect to outer catheter 210, and also so that outer catheter 210 provides support for a distal region of inner catheter 220 to extend further beyond the distal end of outer catheter 210. Outer and inner catheters 210, 220 are depicted showing a distal region of inner catheter 220 extending beyond the distal end of outer catheter 210. As described above, the distance between the distal end of outer catheter 210 and the distal end of inner catheter 220 is configurable such that the distal end of inner catheter 220 can be extended further beyond the distal end of outer catheter 210 after outer catheter 210 is positioned at a desired location in the luminal tissue. Inner catheter 220 can be extended further beyond the distal end of outer catheter 210 by applying a force to the proximal end of inner catheter 220, i.e., by an operator of device 200 pushing a proximal region of inner catheter 220 further into the proximal end of outer catheter 210.

In FIG. 2, the dotted line through the longitudinal axis of inner catheter 220 depicts that inner catheter 220 comprises first inner catheter lumen 221 and second inner catheter lumen 222. First and second inner catheter lumens 221, 222 extend substantially the lateral length of inner catheter 220 and are configured to be fluidically isolated from each other and from outer catheter 210 such that first inner catheter lumen 221 can be filled with fluid and pressurized separately from second inner catheter lumen 222. In some cases, fluid present in first inner catheter lumen 221 is different from fluid present in second inner catheter lumen 222. In some cases, pressure is applied to fluid present in first inner catheter lumen 221 at a different magnitude than pressure applied to fluid present in second inner catheter lumen 222. Separate fluid and pressure sources may be attached to each of first and second inner catheter lumens 221, 222. As described above, any convenient pressure sources may be attached to first and second inner catheter lumens 221, 222, such as, for example, syringes, such as pre-filled syringes. First and second inner catheter lumens 221, 222 may be attached to syringes in any convenient manner, such as, for example, by Luer locks attached to the proximal ends of first and second inner catheter lumens 221, 222.

Present on a distal region of inner catheter 220 that extends beyond the distal end of outer catheter 210 are perforations 240. As described above, perforations 240 are configured to allow fluid to exit first inner catheter lumen 221 in a turbulent flow. Perforations 240 are holes in first inner catheter lumen 221 such that fluid present in first inner catheter lumen 221 may be pressurized to cause such fluid to exit first inner catheter lumen 221 through perforations 240 thereby causing turbulent flow in the luminal tissue as the fluid exits. That is, perforations 240 are used to make first inner catheter lumen 221 in fluidic communication with the luminal tissue in which device 200 is placed. Perforations 240 are present only on first inner catheter lumen 221 such that fluid present in second inner catheter lumen 222 does not exit second inner catheter lumen 222 through perforations 240. As described above, the turbulent flow caused by fluid exiting through perforations 240 is used to identify and pressurize segments of luminal tissue, facilitating isolating segments of luminal tissue, for example, in connection with an endoscopy procedure, such as an ostomy.

Also present on a distal region of inner catheter 220 that extends beyond the distal end of outer catheter 210 is balloon 260. Balloon 260 is depicted as partially inflated in FIG. 2, as the diameter of balloon 260 extends beyond the diameter of outer catheter 210. Balloon 260 is positioned distal to perforations 240 and distal to the distal end of inner catheter 220 and to guidewire passage channel 250. Balloon 250 is configured such that the interior of balloon 260 is in fluidic communication with second inner catheter lumen 222 such that fluid present in second inner catheter lumen 222 may be used to inflate balloon 260. Balloon 260 is configured to be inflated by applying pressure to fluid present in second inner catheter lumen 222. As described above, any convenient pressure source may be used to apply pressure to fluid present in second inner catheter lumen 222, such as, for example, a syringe, such as a pre-filled syringe.

Balloon 260 is configured such that inflating balloon 260 causes the exterior walls of balloon 260 to engage with the luminal tissue, in some cases, occluding a section of the luminal tissue. Balloon 260 may be configured in any convenient shape to facilitate engaging luminal tissue walls, such as, for example, a donut shape or torus shape, as seen in FIG. 2. Using balloon 260 to occlude segments of luminal tissue causes turbulent flow of fluid through perforations 240 to remain proximal to balloon 260 thereby facilitating pressurizing a segment of luminal tissue. In some cases, when a distal region of device 200 is passed over a luminal obstruction, using balloon 260 to occlude segments of luminal tissue causes turbulent flow of fluid through perforations 240 to pressurize a segment of luminal tissue proximal to balloon 260 and distal to such luminal occlusion.

The amount of pressure applied to second inner catheter lumen 222 may vary, depending, for example, on the mechanical characteristics and diameter of the luminal tissue. A pressure source may be attached to second inner catheter lumen 222 that enables gradual increases in pressure. Pressure in balloon 260 may be measured using pressure sensor 295. Pressure sensor 295 may be any convenient pressure sensor and is located on the surface of balloon 260. Pressure sensor 295 may be any convenient, off the shelf pressure sensor, configured to measure pressure inside balloon 260 in any convenient manner. Pressure sensor 295 may be configured to communicate pressure readings to a receiver outside the luminal tissue of a subject in any convenient manner. In some cases, pressure sensor 295 may be connected via a wire, such as a wire threaded through catheter device 200, to a receiver outside the luminal tissue of a subject. In other cases, pressure sensor 295 is configured to wirelessly communicate with a monitor positioned outside the luminal tissue. Device 200 comprises pressure sensor 295 attached to balloon 260 and in addition may comprise a pressure source connected to second inner catheter lumen 222 where device 200 is configured to automatically inflate balloon 260 to a specified pressure measured by pressure sensor 295. That is, a control system, such as an analog or digital control system, may be applied to automatically inflate balloon 260 to a desired pressure, where such pressure is measured using pressure sensor 295, and the results of such measurements are fed back to control the pressure source.

Also present on a distal region of inner catheter 220 that extends beyond the distal end of outer catheter 210 is guidewire passage channel 250. Guidewire passage channel 250 is located near the distal end of inner catheter 220, distal to perforations 240 and distal to balloon 260. Guidewire passage channel 250 is a section of a ring or a loop attached to inner catheter 220 in any convenient manner where the ring or the loop allows guidewire 270 to be threaded through guidewire passage channel 250 and further allows the distal end of device 220 to be guided into a desired position of luminal tissue along guidewire 270. While it need not always be the case, typically guidewire 270 would remain in a fixed or substantially fixed position relative to the luminal tissue and the distal end of device 200 would be advanced along the path set forth by guidewire 270. Guidewire passage channel 250 and its attachment to inner catheter 220 must be sufficiently resilient that pressure applied to device 200 to move device 200 into luminal tissue, for example, into luminal tissue past a luminal obstruction, does not cause guidewire passage channel 250 to separate from inner catheter 220 or guidewire 270 to separate from guidewire passage channel 250. As described above, guidewire 270 may be any convenient, off the shelf, guidewire, such as an endoscopic retrograde cholangiopancreatography (ERCP) wire.

Also present on a distal region of inner catheter 220 that extends beyond the distal end of outer catheter 210 is first radiopaque marker 285. In device 200, first radiopaque marker 285 is located proximal to guidewire passage channel 250 and distal to balloon 260. However, the position of first radiopaque marker 285 on inner catheter 220 may vary in other embodiments. Also present on the distal region of outer catheter 210 is second radiopaque marker 280. Second radiopaque marker 280 may be positioned on outer catheter 210 at a specified distance from the distal end of outer catheter 210, and second radiopaque marker 285 may be positioned on inner catheter 220 a specified distance from the distal end of inner catheter 220. When first and second radiopaque markers 285, 280 are positioned as such, or in a similar manner with reference to fixed features of device 200, the distance between first and second radiopaque markers 280, 285, when imaged using fluoroscopy, such as X-rays, is indicative of a specific segment of luminal tissue, i.e., the luminal tissue present between first and second radiopaque markers 285, 280. First and second radiopaque markers 285, 280 may facilitate isolating segments of luminal tissue when used to identify or characterize segments of luminal tissue present between first and second radiopaque markers 285, 280. As described above, first and second radiopaque markers 285, 280 are configured to be opaque with respect to fluoroscopy, such that, for example, first and second radiopaque markers 285, 280 are visible via X-ray imaging. Each of first and second radiopaque markers 285, 280 may comprise distinguishing markings visible via fluoroscopy such that they can be distinguished from each other under fluoroscopy, i.e., an operator of the device can visualize which aspect of an X-ray image corresponds to which marker. While first and second radiopaque markers 285, 280 are depicted on device 200 in FIG. 2, other embodiments of devices according to the present invention may comprise a plurality of markers that may be placed at fixed distances from each other so that lateral distances in the luminal tissue may be measured by imaging such embodiments of the catheter device under fluoroscopy. As described above, radiopaque markers 285, 280 may further comprise magnets configured to interact with magnets present elsewhere in the luminal tissue or other locations in a subject's anatomy, such as a stomach. Such interaction between magnets may be used to draw different tissues together via the magnetic force between the magnets, such as, for example, drawing luminal tissue closer to stomach tissue in connection with a procedure, such as an ostomy.

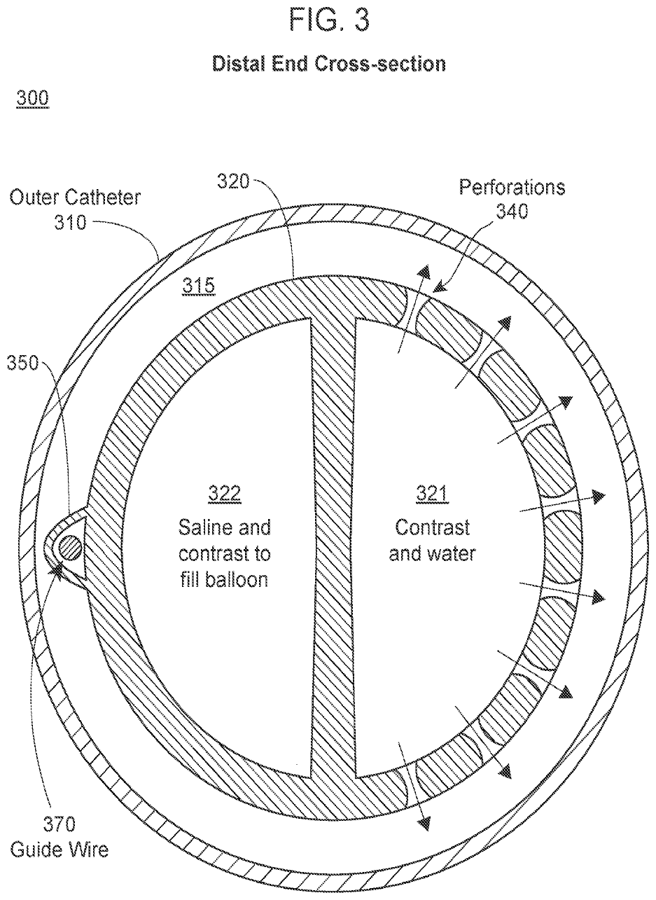

FIG. 3 depicts a cross-sectional view of a distal region of an embodiment of device 300 for isolating luminal tissue according to some aspects of the present disclosure. The distal region of device 300 depicted in FIG. 3 corresponds to a subject-facing region of the device when the device is positioned within luminal tissue, such as gastrointestinal tissue including the jejunum.

Present on device 300 is outer catheter 310. Outer catheter 310 comprises outer catheter lumen 315, in which inner catheter 320 is present. The outer catheter lumen is sized appropriately such that inner catheter 320 fits within the outer catheter lumen, allowing inner catheter 320 to be moved laterally with respect to outer catheter 310, and also so that outer catheter 310 provides support for a distal region of inner catheter 320 to extend further beyond the distal end of outer catheter 310. Outer catheter lumen 315 may be configured with any convenient diameter. The diameter of outer catheter lumen 315 is configured to allow inner catheter 320 to be present in outer catheter 320 and to move laterally within outer catheter 310. The diameter of outer catheter lumen 315 is configured so that the lumen wall of outer catheter 310 offers adequate support for inner catheter 310, including support for a distal region of inner catheter 320 that extends beyond the distal end of outer catheter 310.

Inner catheter 320 present within outer catheter 310 comprises first inner catheter lumen 321 and second inner catheter lumen 322. First inner catheter lumen 321 is fluidically isolated from second inner catheter lumen 322 such that fluid and/or pressure applied to first inner catheter lumen 321 is not present in and/or applied to second inner catheter lumen 322 and vice versa. Fluid present in first inner catheter lumen 321 comprises a first contrast. Fluid present in first inner catheter lumen 321 further comprises water. Fluid present in second inner catheter lumen 322 comprises a second contrast. Fluid present in second inner catheter lumen 322 further comprises a saline. That is, in device 300, fluid present in first inner catheter lumen 321 differs from the fluid present in second inner catheter lumen 322.

In catheter device 300, first and second inner catheter lumens 321, 322 are positioned side-to-side. In other embodiments, the first and second inner catheter lumens may be positioned in a co-axial arrangement. In still other embodiments, the first and second inner catheter lumens comprise separate catheters present within a lumen of the inner catheter (i.e., two separate catheters positioned within the lumen of the inner catheter).

Perforations 340 present on inner catheter fluidically connect first inner catheter lumen 321 with space outside inner catheter 310 (i.e., within the walls of the luminal tissue). As described above, perforations 340 are configured to allow fluid to exit first inner catheter 321 lumen in a turbulent flow. That is, perforations 340 are configured such that when pressure is applied to the fluid present in first inner catheter 321, fluid exists first inner catheter 321 in a turbulent flow. In FIG. 3, arrows show the path of fluid exiting first inner catheter 321 via perforations 340. In device 300, perforations 340 are depicted as forming a hemisphere in cross section. In other embodiments, any convenient configuration of the perforations may be applied.

Guidewire passage channel 350 is present on a distal region of device 300. Guidewire passage channel 350 is shown with a cross-section of guidewire 370 threaded through guidewire passage channel 350. As described above, guidewire passage channel is configured to firmly hold and guide a distal region of device 300 along guidewire 370 to a desired location within the luminal tissue, e.g., a location proximal to a subject's anatomy relevant to an endoscopic procedure. That is, guidewire 370 is typically positioned in a desired location of the luminal tissue of a subject's anatomy prior to positioning the distal region of the catheter device 300 via guidewire guidance into the desired location of the luminal tissue of a subject's anatomy. Guidewire passage channel 350 is formed as a hemisphere affixed or bonded or otherwise attached to the outer wall of inner catheter 320. In other embodiments, the guidewire passage channel may be formed as a ring attached to the outer wall of the inner catheter, or, in other embodiments, the guidewire passage channel is a tube or a section of a tube affixed or bonded or otherwise attached to the outer call of the inner catheter. Guidewire 370 and guidewire passage channel 350 are configured so that guidewire 370 can move laterally within guidewire passage channel 350. While the dimensions of guidewire passage channel 350 and guidewire 370 are not necessarily drawn to scale in FIG. 3, in general, guidewire passage channel 350 and guidewire 370 are smaller in diameter than device 300.

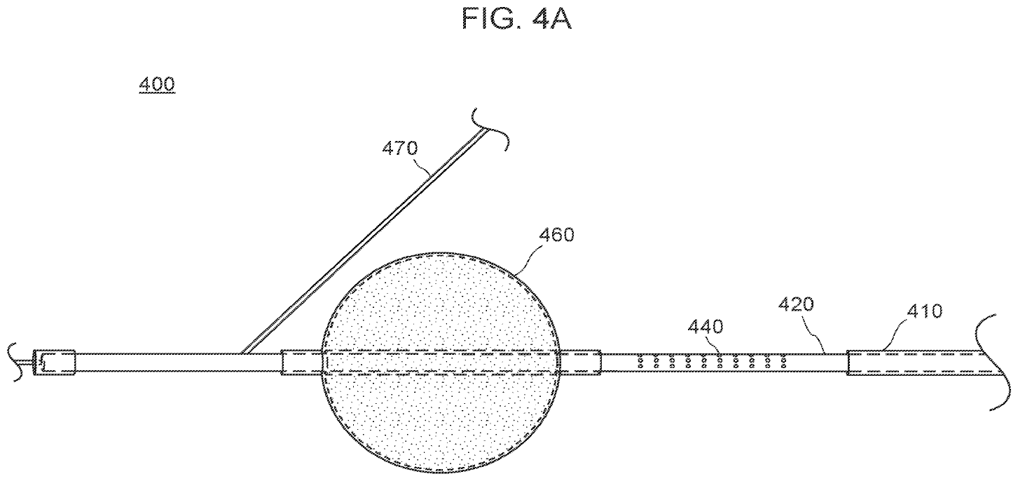

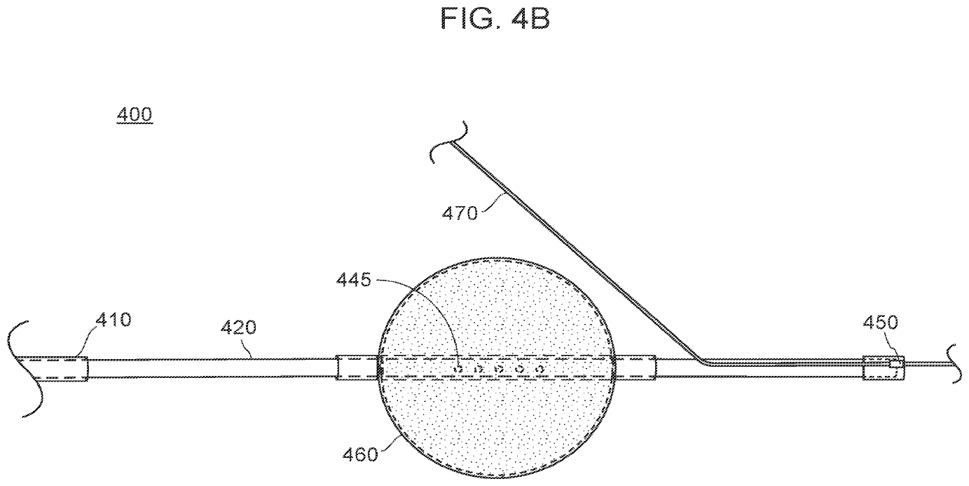

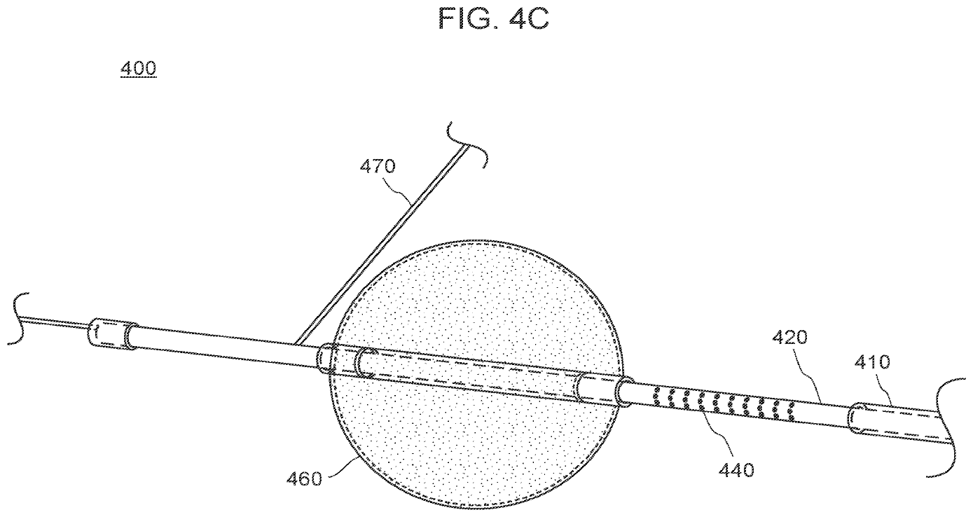

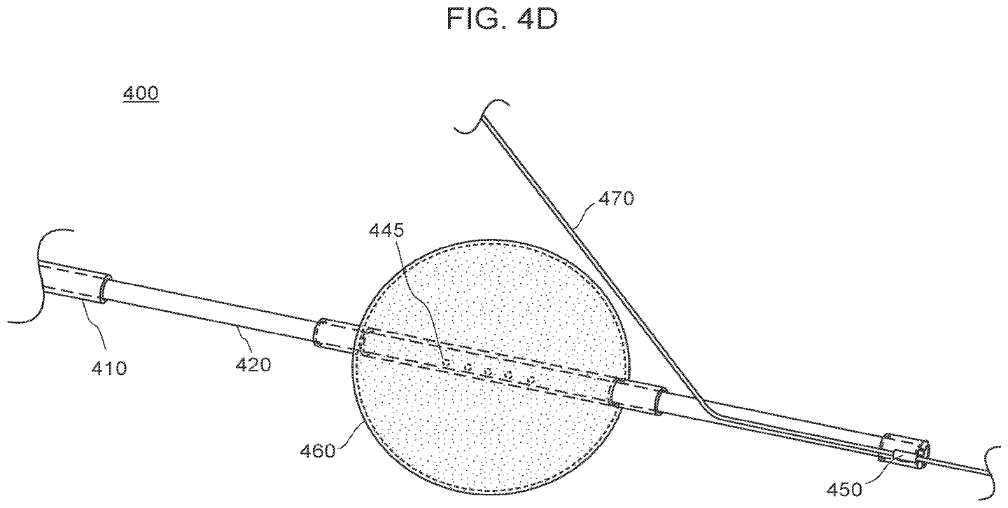

FIGS. 4A-4D depict different views of a distal region of an exemplary embodiment of a device 400 according to the present invention. FIG. 4A depicts a side view of a distal region of device 400, with distal, or subject-facing end on the left side of the figure and proximal, or operator-facing end on the right side of the figure. FIG. 4B depicts a side view of a distal region of device 400, with distal, or subject-facing end on the right side of the figure and proximal, or operator-facing end on the left side of the figure (i.e., FIG. 4B depicts the opposite side of the distal region of device 400 depicted in FIG. 4A). FIG. 4C depicts an isometric view of device 400 with distal, or subject-facing end on the left side of the figure and proximal, or operator-facing end on the right side of the figure. FIG. 4D depicts an isometric view of device 400 with distal, or subject-facing end on the right side of the figure and proximal, or operator-facing end on the left side of the figure (i.e., FIG. 4D depicts an opposite orientation of the distal region of device 400 depicted in FIG. 4C).

Device 400 comprises outer catheter 410, in which inner catheter 420 is present and extends beyond the distal end of outer catheter 410. As described above, inner catheter 410 is configured to move laterally within outer catheter 420. That is, inner catheter 410 is configured so that the distal end of inner catheter 420 can be extended a greater distance beyond the distal end of outer catheter 410. The distal end of inner catheter 410 can be extended as such by, for example, applying force to a proximal region, such as the proximal end, of inner catheter 420 (not shown in FIGS. 4A-4D). In some cases, outer and inner catheters 410, 420 are positioned at a desired location of luminal tissue and inner catheter is then extended further beyond the distal end of outer catheter 410. As depicted in the figure, inner catheter 410 extends beyond the distal end of outer catheter 420 and, as described in detail below, inner catheter 420 comprises several features attached to a distal region of inner catheter 420 that extends beyond the distal end of outer catheter 410. As such, outer catheter 410 is configured to provide support to inner catheter 420, for example, reinforcing the stiffness of inner catheter 420 as it is passed into luminal tissue, for example, passed beyond a luminal obstruction in the luminal tissue, to prevent bending, folding, distorting or otherwise mis-aligning the distal region of device 400.

Inner catheter 420 comprises perforations 440 in a distal region of inner catheter 420. In FIGS. 4A-4D, perforations 440 are present on inner catheter 420 distal to the distal end of outer catheter 420, but in embodiments, may be retracted so that perforations are present on a region of inner catheter 420 within outer catheter 410. As described above, perforations 440 are configured so that fluid present in the first inner catheter lumen (not shown) of inner catheter 420 exits the first inner catheter lumen in a turbulent flow. For example, when pressure is applied to fluid present in the first inner catheter lumen of inner catheter at a proximal region of the first inner catheter lumen, fluid exits perforations 440 in a turbulent flow that can, for example, be visualized using ultrasonic imaging and can create a locally pressurized region of luminal tissue proximal to perforations 440. Perforations 440 may take any convenient shape and configuration. In FIGS. 4A-4D, perforations 440 are arranged in an array extending circumferentially and laterally along inner catheter 420. In other embodiments, the perforations may be arranged in any convenient manner on inner catheter 420.

Also present on inner catheter 420 is balloon 460, located distal to perforations 440. Balloon 460 is depicted in FIGS. 4A-4D as substantially inflated. As described above, balloon 460 may be inflated with fluid present in second inner catheter lumen (not shown) of inner catheter 420. For example, when pressure is applied to fluid present in the second inner catheter lumen of inner catheter 420 at a proximal region of the second inner catheter lumen, fluid fills balloon 460 with fluid exiting holes 445 connecting the second inner catheter lumen and the interior of balloon 460. Balloon 460 may be inflated such that the exterior surface of balloon 460 engages with the luminal tissue in which distal region of device 400 is located. Balloon 460 may be inflated to intentionally cause the balloon to block or occlude a segment of luminal tissue in which a distal region of device 400 is present. Inflating balloon 460 to occlude the luminal tissue may facilitate isolating and/or visualizing a segment of luminal tissue by, for example, causing fluid exiting perforations 440 to exit in a turbulent flow as well as may facilitate causing fluid exiting perforations 440 to pressurize a segment of luminal tissue. In its deflated state, balloon 460 may have a diameter substantially the same as that of inner catheter 420 or outer catheter 410.

In embodiments, the first and second inner catheter lumens are positioned side-by-side within inner catheter 420. This arrangement is illustrated in that FIG. 4A depicts perforations 440 connecting the first inner catheter lumen to the luminal space on one side of device 400, and FIG. 4B depicts holes 445 connecting the second inner catheter lumen with the interior of balloon 460 on the other side of device 400.

Guidewire 470 is configured to guide a distal region of device 400 to a desired location within luminal tissue and is shown connected to a distal region of inner catheter 420 threaded through guidewire passage channel 450. Guidewire passage channel 450 is shown as comprising a plurality of loops affixed to a distal region of inner catheter 420 such that a segment of guidewire 470, when threaded through the loops of guidewire passage channel 450, extends parallel to inner catheter 420.

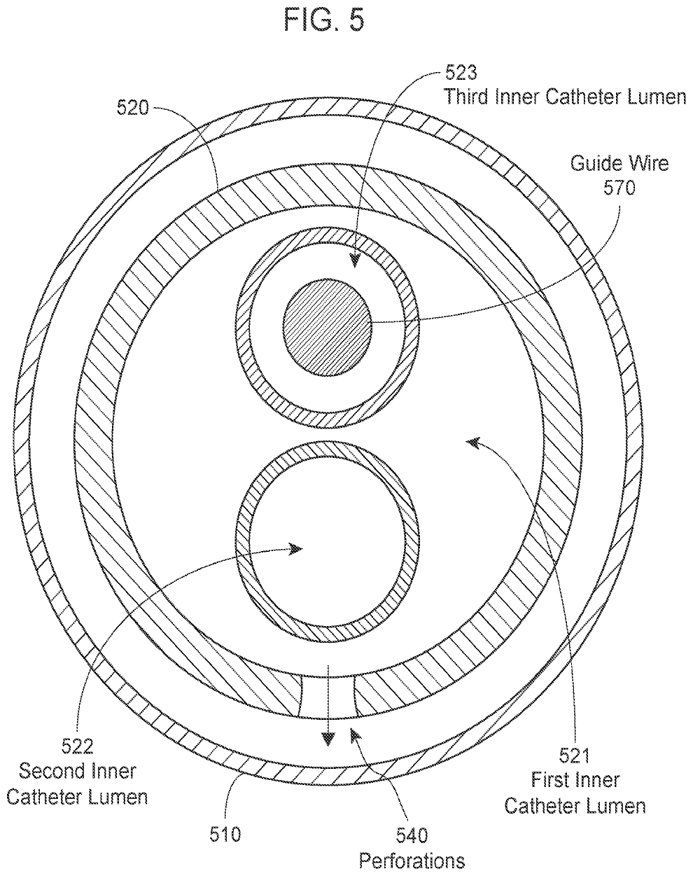

FIG. 5 depicts a cross section of an alternative embodiment of an inner catheter 520 of a catheter device according to the present invention. In the embodiment shown in the figure, inner catheter 520 comprises three inner catheter lumens: first inner catheter lumen 521, connected to perforations 540, is used to generate turbulent flow; second inner catheter lumen 522 is connected to a balloon (not shown) and is used to inflate the balloon by displacing fluid from second inner catheter lumen 522 into the balloon; and third inner catheter lumen 523 is used to hold guidewire 570 (like guidewire 470 seen in FIGS. 4A-4D), for use guiding device to a desired location in the luminal tissue. Inner catheter 520 is present within outer catheter 510.

### Methods for Isolating Luminal Tissue

As summarized above, aspects of the present disclosure include methods for isolating a segment of luminal tissue. Methods according to certain embodiments comprise (a) introducing a catheter device to the luminal tissue, the catheter device comprising: (i) an outer catheter comprising an outer catheter lumen; (iii) an inner catheter positioned within the outer catheter lumen, extending beyond the distal end of the outer catheter, the inner catheter comprising: (A) first and second inner catheter lumens; (B) perforations at a distal region of the inner catheter configured to allow fluid to exit the first inner catheter lumen in a turbulent flow; (C) a guidewire passage channel positioned distal to the perforations, wherein the inner catheter is configured to move laterally within the outer catheter lumen; (iii) a balloon attached to the inner catheter positioned distal to the perforations and proximal to the guidewire passage channel in fluidic communication with the second inner catheter lumen and configured to radially expand; (iv) a guidewire positioned in the guidewire passage channel; (v) a first radiopaque marker on the inner catheter distal to the balloon; and (vi) a second radiopaque marker at a distal region of the outer catheter; (b) generating a turbulent flow at the perforations of the inner catheter by pressurizing fluid present in the first inner catheter lumen; (c) inflating the balloon to isolate a segment of luminal tissue by pressurizing fluid present in the second inner catheter lumen. In other embodiments of the method, the catheter device may comprise any embodiment of the catheter device described above.

### Introducing Catheter to the Luminal Tissue:

In embodiments, introducing a catheter device to the luminal tissue comprises: introducing the guidewire into the luminal tissue, and advancing distal regions of the inner and outer catheters over the guidewire into the luminal tissue. That is, the device is configured to, and the method comprises, advancing a distal region of the device to a desired location of anatomy within the luminal tissue via wire guidance. In other embodiments, introducing a catheter device to the luminal tissue comprises positioning distal regions of the inner and outer catheters in the luminal tissue. In some embodiments, introducing a catheter device to the luminal tissue comprises advancing a distal region of the catheter device past a luminal obstruction.

### Extending Inner Catheter:

In embodiment, the method further comprises moving the inner catheter laterally relative to the outer catheter. In such embodiments the inner catheter is configured to move laterally with respect to the outer catheter. For example, force may be applied to a proximal region of the inner catheter to cause the inner catheter to move laterally with respect to the outer catheter. In other embodiments, the method further comprises positioning a distal region of the catheter device by moving the inner catheter laterally relative to the outer catheter. That is, positioning aspects of the device, such as, for example, the balloon and/or the perforations, may comprise positioning such aspects of the device by moving the inner catheter relative to the outer catheter.

In some embodiments, the catheter device further comprises a locking mechanism, and the method further comprises using the locking mechanism to fix the relative positions of the inner and outer catheters.

In certain embodiments, introducing a catheter device to the luminal tissue comprises: introducing the guidewire into the luminal tissue, advancing distal regions of the inner and outer catheters over the guidewire into the luminal tissue, and extending the inner catheter laterally further beyond the distal end of the outer catheter. In some cases, such embodiments of methods according to the invention further comprise fixing the relative positions of the inner and outer catheters using a locking mechanism.

### Locating the Catheter/Isolated Luminal Segment:

In embodiments, the method further comprises locating the isolated segment of luminal tissue. For example, the method may comprise locating the isolated segment of luminal tissue in connection with performing an endoscopic procedure on or incorporating such isolated segment of luminal tissue. In some cases, the method comprising locating the isolated segment of luminal tissue by locating a distal region of the catheter device. In other cases, the method further comprises locating the isolated segment of luminal tissue using x-ray fluoroscopy. In certain cases, the method further comprises locating a distal region of the catheter device in the luminal tissue using x-ray fluoroscopy. In such embodiments, using x-ray fluoroscopy may comprise imaging the first and second radiopaque markers.

In some cases, the method further comprises locating comprising locating the isolated segment of luminal tissue using ultrasound imaging. In other cases, the method further comprises locating a distal region of the catheter device in the luminal tissue using ultrasound. In such embodiments, using ultrasound may comprise imaging the turbulent flow of fluid exiting the first inner catheter lumen.

### Inflating the Balloon:

In embodiments, the method comprises inflating the balloon to engage the luminal tissue. In such cases, the method may further comprise occluding a section of the luminal tissue by inflating the balloon to engage the luminal tissue.

In some cases, the balloon of the catheter device is torus shaped upon inflation. In certain cases, the balloon comprises an expandable polymer. In other embodiments, inflating the balloon by pressurizing fluid present in the second inner catheter lumen comprises activating a pressure source connected to the proximal end of the second inner catheter lumen. In such embodiments, activating a pressure source may comprise depressing a syringe connected to the proximal end of the second inner catheter lumen.

In embodiments of the method according to the invention, the catheter device further comprises a pressure sensor configured to measure pressure inside the balloon. Any convenient pressure sensor, such as an off-the-shelf pressure sensor, capable of measuring pressure of a catheter balloon while the balloon is present in luminal tissue may be employed. In some cases, the pressure sensor is configured to be present on the luminal tissue. Certain embodiments of the method according to the present invention further comprise wirelessly communicating with the pressure sensor.

In some cases, the method may comprise automatically inflating the balloon using a pressure source and feedback from a pressure sensor configured to turn off or disconnect the pressure source when the balloon reaches a desired pressure. In embodiments of the method, the catheter device further comprises: a pressure source attached to the second inner catheter lumen, and a pressure sensor attached to the balloon, and the method further comprises activating the pressure source to inflate the balloon until the pressure sensor senses that balloon pressure has reached a specified pressure. In embodiments, fluid present in the balloon comprises a contrast and the method further comprises locating a distal region of the catheter device by imaging the contrast in the balloon.

### Generating Turbulent Flow:

In embodiments, generating a turbulent flow at the perforations of the inner catheter by pressurizing fluid present in the first inner catheter lumen comprises activating a pressure source connected to the proximal end of the first inner catheter lumen. Any convenient pressure source may be applied, such as, for example, a syringe, such as a pre-filled syringe, such that activating a pressure source comprises depressing a syringe connected to the proximal end of the first inner catheter lumen.

In other embodiments, the method further comprises pressurizing segments of luminal tissue with the turbulent flow at the perforations of the inner catheter. In still other embodiments, fluid present in the first inner catheter lumen comprises a contrast, and the method further comprises locating a distal region of the catheter device by imaging the contrast in the fluid exiting the first inner catheter lumen in the turbulent flow.

### Methods Using Variations of Catheter Devices:

In embodiments, the method further comprises entering an endoscope over the catheter device. Any convenient endoscope may be employed, such as standard, off-the-shelf endoscopes used in endoscopic procedures, such as endoscopic procedures involving gastrointestinal tissue. Other embodiments further comprise passing the catheter device through a working channel of a therapeutic upper endoscope. Still other embodiments further comprise imaging using a camera present on the catheter device. As described above, any convenient camera, such as those conventionally used in endoscopic procedures may be applied. Certain other embodiments further comprise applying a needle present on the catheter device. As described above, any convenient needle, such as a needle configured to puncture luminal tissue or a luminal obstruction may be employed.

### Luminal Tissue:

In embodiments, the luminal tissue comprises enteral tissue. In some embodiments, the luminal tissue comprises a gastrointestinal lumen.

In certain embodiments, the luminal tissue is luminal tissue of a subject. In such embodiments, the subject may be human. In other embodiments, the subject may be an animal that is not human (that is, in “non-human subjects”) such as, but not limited to, birds, mice, rats, dogs, cats, livestock and horses.

### Applications of Method:

In embodiments, the method is a method for treating a subject for Malignant Gastric Outlet Obstruction (MGOO). In some embodiments, the method is a method for treating a subject for benign gastric outlet obstruction. In other embodiments, the method is a method for treating a subject affected by complete or partial obstruction of luminal gastrointestinal tract. In still other embodiments, the method is a method for treating a subject by creating an ostomy or fistula in the subject.

In certain embodiments, the method is a method for treating a subject by facilitating therapeutic interventional endoscopy. In some cases, the method is a method for treating a subject by facilitating placement of a lumen-apposing metal stent (LAMS) in a subject. In other cases, the method is a method for treating a subject by facilitating an endoscopic ultrasound guided gastroenterostomy (EUS-GE) procedure. In still other cases, the method is a method for treating a subject by facilitating an endoscopic gastrojejunostomy procedure.

As described above, the subject may be a human or a non-human animal. A human subject may be male or female and may be any age, such as adult, toddler, juvenile, child, etc. While the subject may be of any body type or body size, in some instances, the subject does not exhibit normal body mass index, such as an underweight, overweight or obese subject.

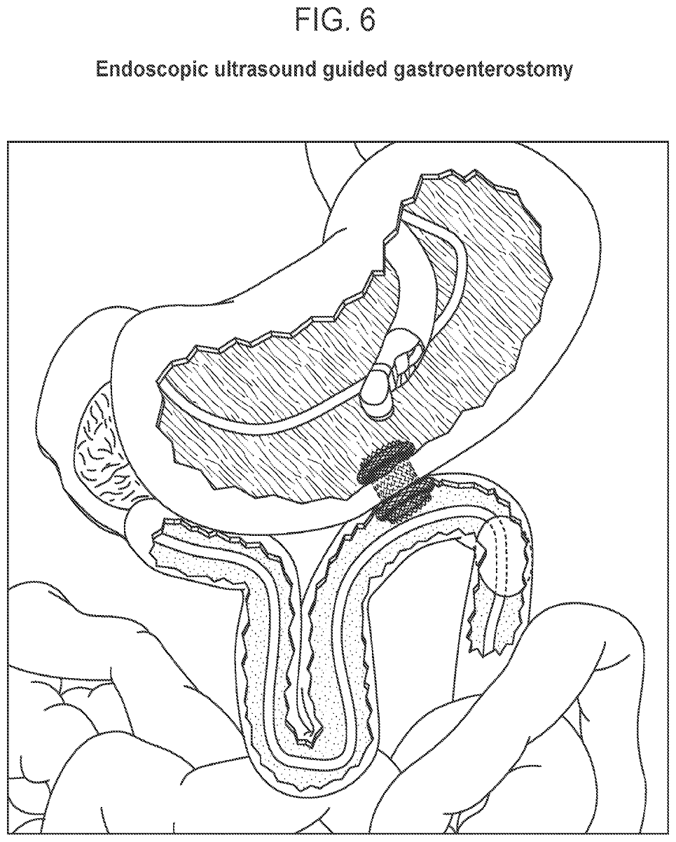

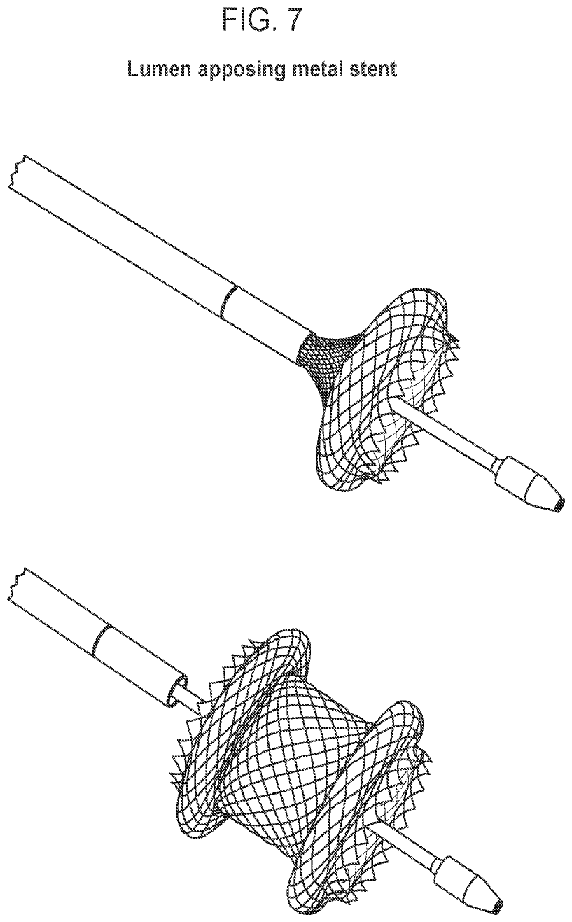

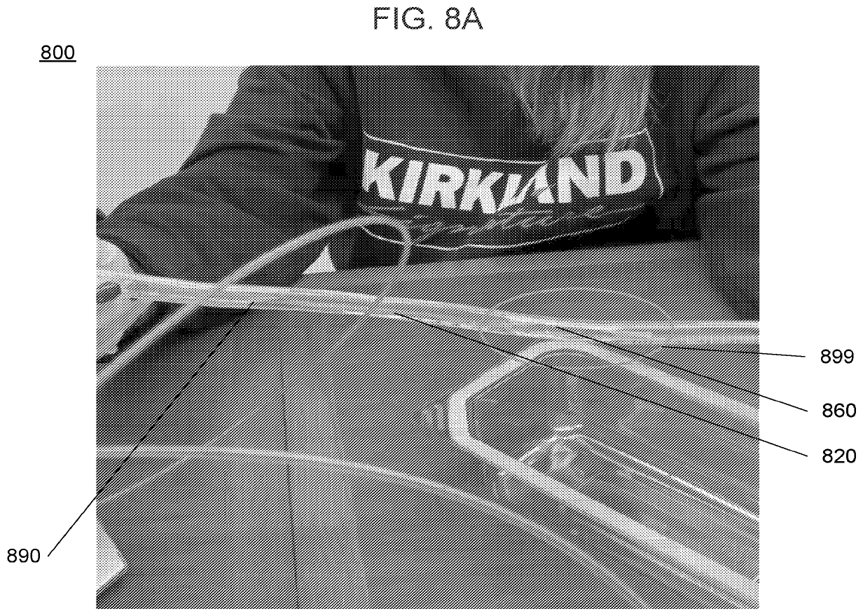

FIG. 6 depicts cutaway view of a stomach and gastrointestinal tissue of a subject to illustrate an example of tissues on which devices and methods described herein may be applied. Luminal tissue, such as that depicted in FIG. 6, may comprise a luminal obstruction. Embodiments of the present invention may relate to passing devices via wire guidance through such tissue for use isolating segments of luminal tissue, for example, in connection with an endoscopic procedure, such as an endoscopic ultrasound guided gastroenterostomy (EUS-GE) procedure in a subject with Malignant Gastric Outlet Obstruction (MGOO). FIG. 7 depicts a lumen apposing metal stent in both a compressed and expanded state. As described above embodiments of the present invention may be configured to facilitate placement of a lumen-apposing metal stent (LAMS), such as that shown in FIG. 7, in luminal tissue of a subject. In some cases, the method is a method for treating a subject by facilitating placement of a lumen-apposing metal stent (LAMS) in a subject.

### Utility

The subject devices and methods find use in a variety of applications where it is desirable to isolate a segment of luminal tissue. Further, the subject devices and methods find use in applications where it is desirable to isolate, visualize, identify and manipulate a segment of luminal tissue. In some embodiments, the methods and devices described herein find use in clinical settings such as any clinical setting where traditional endoscopic or surgical-based gastrointestinal interventions may be applied, such as those procedures designed to address or alleviate luminal obstructions, such as obstructions in gastrointestinal tissue. For example, embodiments of the devices methods described herein find use in alleviating luminal obstructions related to Malignant Gastric Outlet Obstruction (MGOO). Further, embodiments facilitate the adoption, including the adoption in non-academic clinics, of endoscopic procedures such as endoscopic ultrasound guided gastroenterostomy (EUS-GE) procedure. In addition, the subject methods and devices find use in accessing, via catheter, luminal tissue distal to sever luminal obstructions, as well as improving the effectiveness and accuracy of isolating luminal tissue, such as by visualizing luminal tissue via radiopaque markers and/or turbulent flow of fluid. In some cases, the subject methods and devices find use in improving ease of operation of catheter devices for endoscopic procedures.

The following is offered by way of illustration and not by way of limitation.

### EXPERIMENTAL

### Benchtop Testing:

Various design aspects of embodiments of catheter devices of the present invention were explored in the context of benchtop testing. In particular, various designs of the distal end of embodiments of catheter devices of the present invention were prototyped and tested to study such embodiments' functionality from the standpoint of: (1) occluding and isolating a section of intestine and (2) enhancing ultrasonic characterization of an occluded section of intestine. Prototype catheter devices of the present invention were developed based on the embodiments depicted in FIGS. 2-3.

To study intestinal occlusion and isolation using prototype catheter devices, prototype catheter devices were tested using bench models of intestinal segments. Balloons of prototype catheter devices were inflated via the second inner catheter lumen and the degree of occlusion of the intestinal segment was characterized. Similarly, the functionality of the perforations was confirmed by filling the first inner catheter lumen with fluid, pressurizing the fluid present within such lumen and characterizing the flow rates (volume per unit time) of fluid exiting the first inner catheter lumen through each perforation.

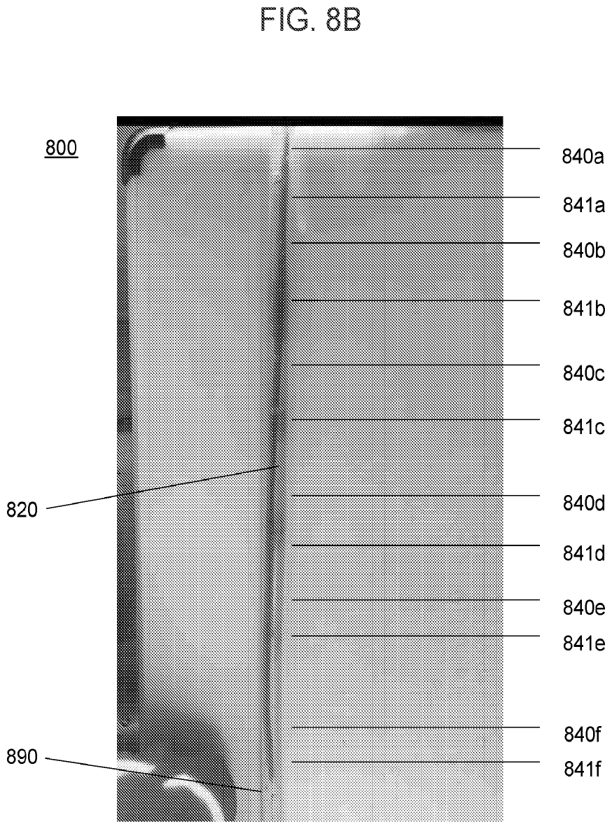

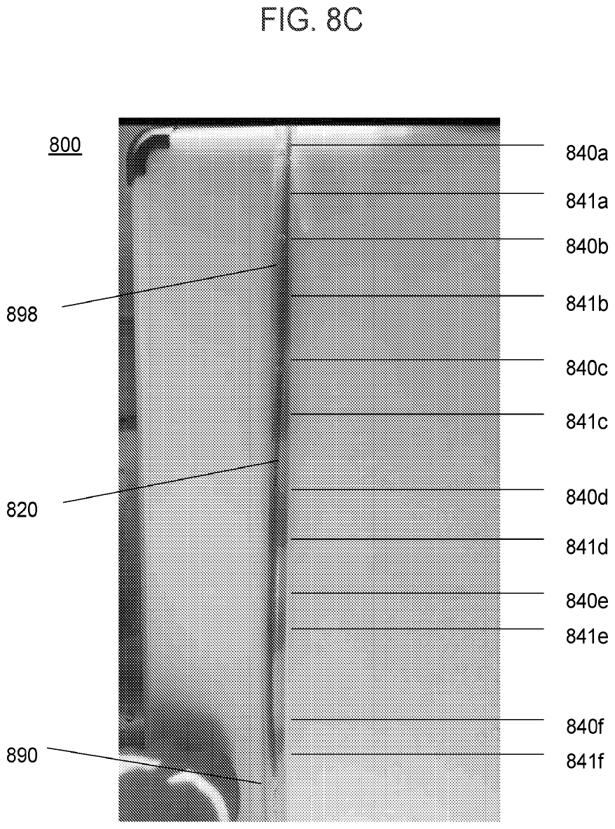

FIG. 8A shows a representative example of testing as described above. Testing environment 800 comprises a modeled small bowel segment 890, comprising clear plastic tubing, within which a prototype catheter device of the present invention is positioned, with the balloon 860 within circled highlighted region 899. The tubing in testing environment 800 is clear plastic tubing (uxcell PVC Clear Vinyl Tubing) with a 6 mm inner diameter tube. The prototype catheter device used in this study comprises balloons designed to expand to a maximum diameter of 8 mm. The perforations of inner catheter 820 as well as the first and second inner catheter lumen are not visible in FIG. 8A. Balloon 860 was inflated and water within the first inner catheter lumen was then pressurized using a syringe pump, causing water to flow into the modeled bowel segment via the perforations. The observation that water is only present directly to the left of circled highlighted region 899 (i.e., in a proximal direction relative to balloon 860) and that water is not present in the region distal to balloon 860 reflects complete successful occlusion of the modeled bowel segment 860 by inflation of the balloon of the prototype catheter device.

FIGS. 8B-C show the results of additional testing conducted in testing environment 800 designed to allow for to precisely characterize fluid flow from inner catheter 820 through the perforations 840. The same view of testing environment 800 is seen in FIGS. 8B-C and comprises modeled bowel segment 890, within which a prototype catheter device of the present invention is positioned. The prototype catheter device is oriented such that the top of the figure corresponds to a more distal direction and the bottom of the figure corresponds to a more proximal direction. The distal region of the prototype catheter device comprises distal region of inner catheter 820 as well as perforations 840a, 840b, 840c, 840d, 840e, 840f. Also seen in FIGS. 8B-C are regions 841a, 841b, 841c, 841d, 841e, 841f within plastic balloon 890 that are immediately proximal to each perforation 840a, 840b, 840c, 840d, 840e, 840f. The balloon of the prototype catheter device is not shown in FIGS. 8B-C. Perforations 840a, 840b, 840c, 840d, 840e, 840f are spaced at the following intervals: 1.5 cm, 3.75 cm, 6 cm, 8.25 cm, 10.5 cm, respectively. Collectively, the perforation array spans a length of 30 cm. Taking into account the spatial resolution of standard endoscopic ultrasound transducers, enhancing echogenicity of a 30 cm length of small bowel can greatly facilitate successful accomplishment of interventional procedures

In connection with FIGS. 8B-C, after first inflating the balloon to occlude the modeled bowel segment 890 as described above, test fluid comprising water with food coloring within the inner catheter lumen is pressurized using a syringe pump, causing test fluid to flow into the modeled bowel segment via the perforations. FIG. 8B and FIG. 8C show test environment 800 at t=0.5 seconds and t=1 second after pressurization of the test fluid within the inner lumen. The flow of test fluid through perforations 840a, 840b, 840c, 840d, 840e, 840f is dependent on perforation position, with the flow rate through the most distal perforation, perforation 840a, approximately twice that of the flow rate through the most proximal perforation, perforation 840f, as seen in FIG. 8B and FIG. 8C. The observed flows through the perforation array is consistent with improved echogenicity of a bowel segment.

### In Vivo Testing:

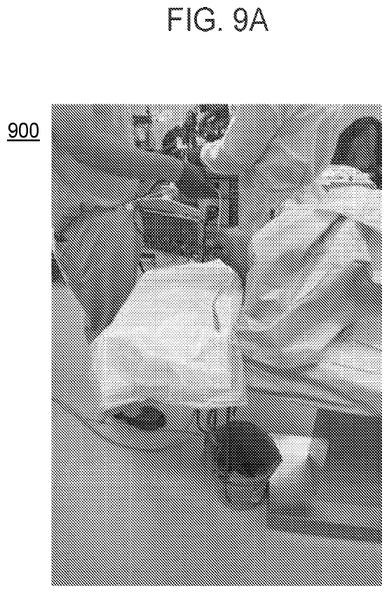

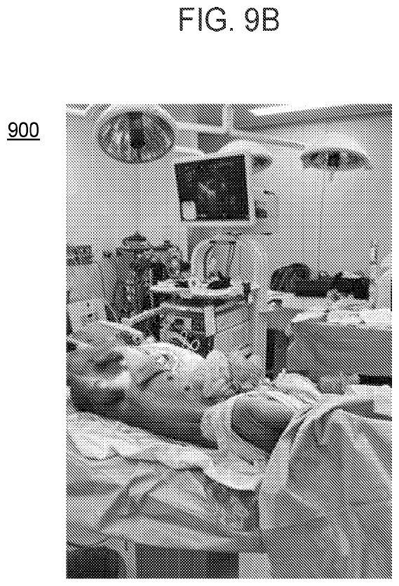

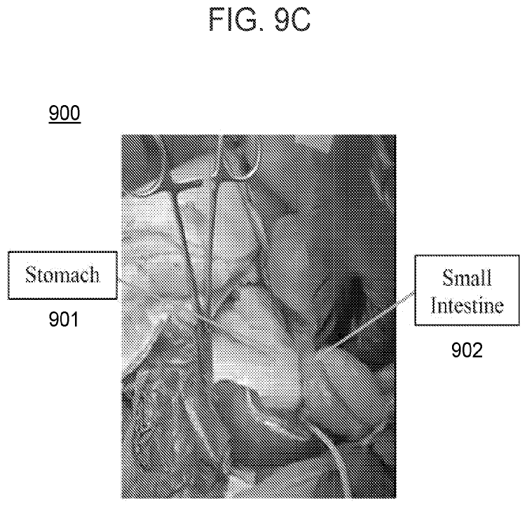

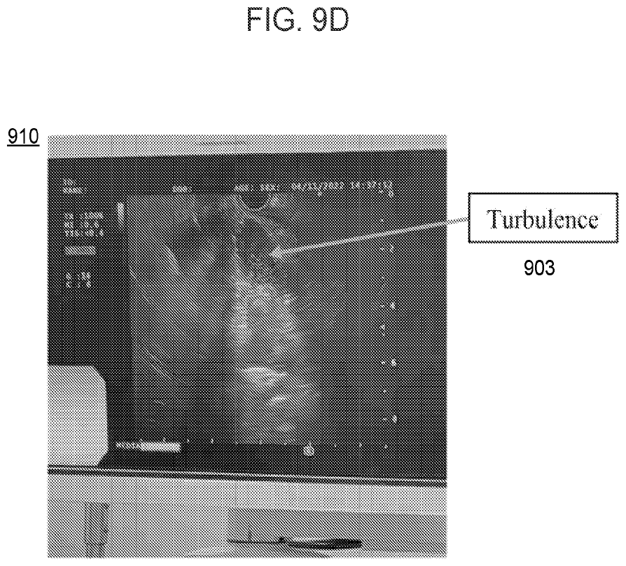

Various design aspects of embodiments of catheter devices of the present invention were explored in the context of in vivo testing. In particular, various designs of catheter devices of the present invention were prototyped, tested and evaluated on a pig carcass. Specifically, in vivo testing of a prototype catheter device of the present invention was used to visualize the turbulent flow of water exiting from the inner catheter perforations via ultrasound visualization of the catheter device in vivo. FIGS. 9A-C show such in vivo testing environment 900. Prototypes of catheter devices were of lengths ranging from 180 and 240 cm in order to allow sufficient length to track through the digestive tract to reach the target region. Both compliant and noncompliant balloons were used successfully on prototype catheter devices of the present invention, as well as both relatively longer and relatively shorter balloon lengths, so long as the balloon was capable of blocking off the distal end of the target section of the small intestine. Certain prototypes comprised a stereolithography SLA resin printed cap with an external guidewire rail loop.

In an endoscopic gastrojejunostomy procedure, fluoroscopy is utilized for visualization, and therefore a contrast solution would need to be ejected from the inner catheter perforations of the catheter device. For the purposes of simplifying this in vivo study, water was utilized to visualize the turbulence under ultrasound. A result of ultrasound imaging 910 is shown in FIG. 9D.

Multiple catheter devices comprising different inner catheter diameters, orientation of perforations as well as balloon diameters were utilized in connection with in vivo testing on the pig carcass.

A thoracotomy was performed on the pig carcass to open the chest to better enable access the stomach 901 and small intestine 902. In addition, the small intestine was positioned against the wall of the stomach to create an environment that more closely resembled human anatomy, as shown in FIG. 9C. Additionally, an insertion hole was created to allow catheter prototypes to enter directly into the stomach and be manually pushed down the small intestine to reduce set up time.

FIG. 9D depicts an ultrasound image 910 showing how fluid flow through the catheters' perforations manifested in ultrasound visualization. In the ultrasound image 910, the flow in the bowel segment appears to be swirling and turbulent. Through studies of prototype devices with the 30 cm long perforation arrays as described above along with prototype devices with shorter perforation arrays, visualization improvement was greatest with prototype devices with 5 cm long perforation arrays, as fluid flows through the perforation arrays generated swirling and an appearance of turbulence on the ultrasound display.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

What is claimed is:

1. A catheter device for isolating segments of luminal tissue, the device comprising:
(a) an outer catheter comprising an outer catheter lumen;
(b) an inner catheter positioned within the outer catheter lumen, extending beyond the distal end of the outer catheter, the inner catheter comprising:
(i) first and second inner catheter lumens;
(ii) perforations at a distal region of the inner catheter configured to allow fluid to exit the first inner catheter lumen in a turbulent flow; and
(iii) a guidewire passage channel positioned distal to the perforations,
wherein the inner catheter is configured to move laterally within the outer catheter lumen;
(c) a balloon attached to the inner catheter positioned distal to the perforations and proximal to the guidewire passage channel in fluidic communication with the second inner catheter lumen and configured to radially expand;
(d) a guidewire positioned in the guidewire passage channel;
(e) a first radiopaque marker on the inner catheter distal to the balloon; and
(f) a second radiopaque marker at a distal region of the outer catheter.

2. The catheter device according to any of the previous claims, wherein the device is configured to pressurize segments of the luminal tissue with the turbulent flow of fluid exiting the first inner catheter lumen.

3. The catheter device according to any of the previous claims, wherein the device is configured so that the turbulent flow of fluid exiting the first inner catheter lumen identifies a distal segment of luminal tissue for creation of an ostomy.

4. The catheter device according to any of the previous claims, wherein the balloon is configured to engage the luminal tissue to occlude a section of the luminal tissue upon inflation.

5. The catheter device according to any of the previous claims, wherein the balloon is configured to be inflated with fluid present in the second inner catheter lumen.

6. The catheter device according to any of the previous claims, wherein the balloon is torus shaped upon inflation.

7. The catheter device according to any of the previous claims, wherein the balloon comprises an expandable polymer.

8. The catheter device according to any of the previous claims, further comprising:
a pressure source attached to the second inner catheter lumen; and
a pressure sensor attached to the balloon,
wherein the pressure source and pressure sensor are configured to automatically inflate the balloon to a specified pressure.

9. The catheter device according to any of the previous claims, further comprising a first pressure source configured inflate the balloon.

10. The catheter device according to claim 9, wherein the first pressure source is in fluidic communication with the first inner catheter lumen and the balloon.

11. The catheter device according to any of claims 9 to 10, wherein the first pressure source is a syringe.

12. The catheter device according to any of the previous claims, further comprising a second pressure source configured to cause fluid to exit the first inner catheter lumen through the perforations in a turbulent flow.

13. The catheter device according to claim 12, wherein the second pressure source is in fluidic communication with the second inner catheter lumen.

14. The catheter device according to any of claims 12 to 13, wherein the second pressure source is a syringe.

15. The catheter device according to any of the previous claims, wherein fluid present in the first inner catheter lumen comprises a first contrast.

16. The catheter device according to claim 15, wherein fluid present in the first inner catheter lumen further comprises water.

17. The catheter device according to any of the previous claims, wherein fluid present in the second inner catheter lumen comprises a second contrast.

18. The catheter device according to claim 17, wherein fluid present in the first inner catheter lumen further comprises a saline solution.

19. The catheter device according to any of the previous claims, wherein the outer catheter comprises a polymeric material.

20. The catheter device according to any of the previous claims, wherein the outer catheter comprises a diameter of 10 Fr.

21. The catheter device according to any of the previous claims, wherein the outer catheter comprises a length of between 200 to 400 cm.

22. The catheter device according to any of the previous claims, wherein the outer catheter lumen is configured to accommodate a 7 Fr inner catheter present in the outer catheter lumen.

23. The catheter device according to any of the previous claims, further comprising a locking mechanism configured to fix a length at which the inner catheter extends beyond the distal end of the outer catheter.

24. The catheter device according to claim 23, wherein the locking mechanism is configured to indicate the length at which the inner catheter extends beyond the distal end of the outer catheter.

25. The catheter device according to any of claims 23 to 24, wherein the locking mechanism is present on the outer catheter.

26. The catheter device according to any of claims 23 to 25, wherein the locking mechanism is present at a proximal region of the outer catheter.

27. The catheter device according to any of claims 23 to 26, wherein the device is configured so the locking mechanism is positioned external to the luminal tissue.

28. The catheter device according to any of the previous claims, wherein the inner catheter comprises a polymeric material.

29. The catheter device according to any of the previous claims, wherein the inner catheter comprises a length of between 0 to 100 cm.

30. The catheter device according to any of the previous claims, wherein the inner catheter extends distal to the balloon by 5 to 20 cm.

31. The catheter device according to any of the previous claims, wherein lateral movement of the inner catheter within the outer catheter lumen comprises extending the distal end of the inner catheter further beyond the distal end of the outer catheter.

32. The catheter device according to any of the previous claims, wherein the inner catheter is configured to move laterally within the outer catheter lumen by 0 to 50 cm.

33. The catheter device according to any of the previous claims, wherein lateral movement of the inner catheter is controlled by manipulating the inner catheter at a proximal region of the device.

34. The catheter device according to any of the previous claims, wherein the first and second inner catheter lumens are positioned side by side.

35. The catheter device according to any of the previous claims, wherein the first and second inner catheter lumens are coaxial.

36. The catheter device according to any of the previous claims, wherein distal ends of the first and second inner catheters comprise fixed or removeable Luer locks.

37. The catheter device according to any of the previous claims, further comprising a third inner catheter lumen.

38. The catheter device according to claim 37, wherein the guidewire is present in the third inner catheter lumen.

39. The catheter device according to any of the previous claims, wherein the guidewire and the guidewire passage channel are configured to direct distal regions of the inner and outer catheters to a position in the luminal tissue.

40. The catheter device according to any of the previous claims, wherein the device is configured to be advanced over the guidewire past a luminal obstruction.

41. The catheter device according to any of the previous claims, wherein the guidewire is configured to remain in a fixed position relative to the luminal tissue and the inner and outer catheters move laterally within the luminal tissue.

42. The catheter device according to any of the previous claims, wherein the guidewire is an endoscopic retrograde cholangiopancreatography (ERCP) wire.

43. The catheter device according to any of the previous claims, wherein the guidewire comprises a diameter of 0.035″ or less.

44. The catheter device according to any of the previous claims, further comprising a pressure sensor configured to measure pressure inside the balloon.

45. The catheter device according to claim 44, wherein the pressure sensor is positioned on the surface of the balloon.

46. The catheter device according to any of claims 44 to 45, wherein the pressure sensor is configured to wirelessly communicate with a monitor positioned outside the luminal tissue.

47. The catheter device according to any of the previous claims, wherein the device is configured for use with x-ray fluoroscopy.

48. The catheter device according to any of the previous claims, wherein the device is configured for use with endoscopic ultrasound.

49. The catheter device according to any of the previous claims, further comprising additional radiopaque markers.

50. The catheter device according to any of the previous claims, wherein the radiopaque markers are configured to mark specified distances on the inner catheter.

51. The catheter device according to any of the previous claims, wherein the first radiopaque marker comprises a magnet.

52. The catheter device according to claim 51, wherein the first radiopaque marker is configured to oppose a second magnet present on an anastomosis device.

53. The catheter device according to claim 52, wherein the first radiopaque marker and the second magnet are configured to draw the luminal tissue closer to tissue where the anastomosis device is present.

54. The catheter device according to any of claims 52 to 53, wherein the first radiopaque marker and the second magnet are configured to draw jejunal tissue closer to stomach tissue.

55. The catheter device according to any of the previous claims, further comprising a detachable handle positioned at the proximal end of the device and configured to allow an endoscope to be entered and removed over the outer catheter.

56. The catheter device according to any of the previous claims, further comprising a handle configured to allow passage through a working channel of a therapeutic upper endoscope.

57. The catheter device according to claim 56, wherein the handle is configured to be under 3.7 mm in diameter.

58. The catheter device according to any of the previous claims, further comprising a camera present at a distal region of the device.

59. The catheter device according to any of the previous claims, further comprising a needle present at a distal region of the device.

60. The catheter device according to any of the previous claims, wherein the luminal tissue comprises enteral tissue.

61. The catheter device according to any of the previous claims, wherein the luminal tissue comprises a gastrointestinal lumen.

62. The catheter device according to any of the previous claims, wherein the luminal tissue is luminal tissue of a subject.

63. The catheter device according to any of the previous claims, wherein the subject is human.

64. The catheter device according to any of the previous claims, wherein the device is configured to treat a subject for Malignant Gastric Outlet Obstruction (MGOO).

65. The catheter device according to any of the previous claims, wherein the device is configured to treat a subject for benign gastric outlet obstruction.

66. The catheter device according to any of the previous claims, wherein the device is configured to treat a subject affected by complete or partial obstruction of luminal gastrointestinal tract.

67. The catheter device according to any of the previous claims, wherein the device is configured to facilitate creation an ostomy or fistula in a subject.

68. The catheter device according to any of the previous claims, wherein the device is configured to facilitate therapeutic interventional endoscopy in a subject.

69. The catheter device according to any of the previous claims, wherein the device is configured to facilitate placement of a lumen-apposing metal stent (LAMS) in a subject.

70. The catheter device according to any of the previous claims, wherein the device is configured to facilitate an endoscopic ultrasound guided gastroenterostomy (EUS-GE) procedure in a subject.

71. The catheter device according to any of the previous claims, wherein the device is configured to facilitate an endoscopic gastrojejunostomy procedure in a subject.

72. The catheter device according to any of claims 64 to 71, wherein the subject is a human subject.

73. A method for isolating a segment of luminal tissue, the method comprising:
(a) introducing a catheter device to the luminal tissue, the catheter device comprising:
(i) an outer catheter comprising an outer catheter lumen;
(iii) an inner catheter positioned within the outer catheter lumen, extending beyond the distal end of the outer catheter, the inner catheter comprising:
(A) first and second inner catheter lumens;
(B) perforations at a distal region of the inner catheter configured to allow fluid to exit the first inner catheter lumen in a turbulent flow;
(C) a guidewire passage channel positioned distal to the perforations,
wherein the inner catheter is configured to move laterally within the outer catheter lumen;
(iii) a balloon attached to the inner catheter positioned distal to the perforations and proximal to the guidewire passage channel in fluidic communication with the second inner catheter lumen and configured to radially expand;
(iv) a guidewire positioned in the guidewire passage channel;
(v) a first radiopaque marker on the inner catheter distal to the balloon; and
(vi) a second radiopaque marker at a distal region of the outer catheter;
(b) generating a turbulent flow at the perforations of the inner catheter by pressurizing fluid present in the first inner catheter lumen;
(c) inflating the balloon to isolate a segment of luminal tissue by pressurizing fluid present in the second inner catheter lumen.

74. The method according to claim 73, wherein introducing a catheter device to the luminal tissue comprises:
introducing the guidewire into the luminal tissue; and
advancing distal regions of the inner and outer catheters over the guidewire into the luminal tissue.

75. The method according to any of claims 73 to 74, wherein introducing a catheter device to the luminal tissue comprises positioning distal regions of the inner and outer catheters in the luminal tissue.

76. The method according to any of claims 73 to 75, wherein introducing a catheter device to the luminal tissue comprises advancing a distal region of the catheter device past a luminal obstruction.

77. The method according to any of claims 73 to 76, further comprising moving the inner catheter laterally relative to the outer catheter.

78. The method according to any of claims 73 to 77, further comprising positioning a distal region of the catheter device by moving the inner catheter laterally relative to the outer catheter.

79. The method according to any of claims 73 to 78,
wherein the catheter device further comprises a locking mechanism, and
wherein the method further comprises using the locking mechanism to fix the relative positions of the inner and outer catheters.

80. The method according to any of claims 73 to 79, wherein introducing a catheter device to the luminal tissue comprises:
introducing the guidewire into the luminal tissue;
advancing distal regions of the inner and outer catheters over the guidewire into the luminal tissue; and
extending the inner catheter laterally further beyond the distal end of the outer catheter.

81. The method according to claim 80, further comprising fixing the relative positions of the inner and outer catheters using a locking mechanism.

82. The method according to any of claims 73 to 81, further comprising locating the isolated segment of luminal tissue.

83. The method according to any of claims 73 to 82, further comprising locating the isolated segment of luminal tissue by locating a distal region of the catheter device.

84. The method according to any of claims 73 to 83, further comprising locating the isolated segment of luminal tissue using x-ray fluoroscopy.

85. The method according to any of claims 73 to 84, further comprising locating the isolated segment of luminal tissue using ultrasound imaging.

86. The method according to any of claims 73 to 85, further comprising locating a distal region of the catheter device in the luminal tissue using x-ray fluoroscopy.

87. The method according to claim 86, wherein using x-ray fluoroscopy comprises imaging the first and second radiopaque markers.

88. The method according to any of claims 73 to 87, further comprising locating a distal region of the catheter device in the luminal tissue using ultrasound.

89. The method according to claim 88, wherein using ultrasound comprises imaging the turbulent flow of fluid exiting the first inner catheter lumen.

90. The method according to any of claims 73 to 89, further comprising inflating the balloon to engage the luminal tissue.

91. The method according to claim 90, further comprising occluding a section of the luminal tissue by inflating the balloon to engage the luminal tissue.

92. The method according to any of claims 73 to 91, wherein the balloon is torus shaped upon inflation.

93. The method according to any of claims 73 to 92, wherein the balloon comprises an expandable polymer.

94. The method according to any of claims 73 to 93, wherein inflating the balloon by pressurizing fluid present in the second inner catheter lumen comprises activating a pressure source connected to the proximal end of the second inner catheter lumen.

95. The method according to claim 94, wherein activating a pressure source comprises depressing a syringe connected to the proximal end of the second inner catheter lumen.

96. The method according to any of claims 73 to 95, wherein the catheter device further comprises a pressure sensor configured to measure pressure inside the balloon.

97. The method according to claim 96, further comprising wirelessly communicating with the pressure sensor.

98. The method according to any of claims 73 to 97,
wherein the catheter device further comprises:
a pressure source attached to the second inner catheter lumen; and
a pressure sensor attached to the balloon, and
wherein the method further comprises activating the pressure source to inflate the balloon until the pressure sensor senses that balloon pressure has reached a specified pressure.

99. The method according to any of claims 73 to 98,
wherein fluid present in the balloon comprises a contrast, and
wherein the method further comprises locating a distal region of the catheter device by imaging the contrast in the balloon.

100. The method according to any of claims 73 to 99, wherein generating a turbulent flow at the perforations of the inner catheter by pressurizing fluid present in the first inner catheter lumen comprises activating a pressure source connected to the proximal end of the first inner catheter lumen.

101. The method according to claim 100, wherein activating a pressure source comprises depressing a syringe connected to the proximal end of the first inner catheter lumen.

102. The method according to any of claims 73 to 101, further comprising pressurizing segments of luminal tissue with the turbulent flow at the perforations of the inner catheter.

103. The method according to any of claims 73 to 102,
wherein fluid present in the first inner catheter lumen comprises a contrast, and
wherein the method further comprises locating a distal region of the catheter device by imaging the contrast in the fluid exiting the first inner catheter lumen in the turbulent flow.

104. The method according to any of claims 73 to 103, further comprising entering an endoscope over the catheter device.

105. The method according to any of claims 73 to 104, further comprising passing the catheter device through a working channel of a therapeutic upper endoscope.

106. The method according to any of claims 73 to 105, further comprising imaging using a camera present on the catheter device.

107. The method according to any of claims 73 to 106, further comprising applying a needle present on the catheter device.

108. The method according to any of claims 73 to 107, wherein the luminal tissue comprises enteral tissue.

109. The method according to any of claims 73 to 108, wherein the luminal tissue comprises a gastrointestinal lumen.

110. The method according to any of claims 73 to 109, wherein the luminal tissue is luminal tissue of a subject.

111. The method according to claim 110, wherein the subject is human.

112. The method according to any of claims 73 to 111, wherein the method is a method for treating a subject for Malignant Gastric Outlet Obstruction (MGOO).

113. The method according to any of claims 73 to 112, wherein the method is a method for treating a subject for benign gastric outlet obstruction.

114. The method according to any of claims 73 to 113, wherein the method is a method for treating a subject affected by complete or partial obstruction of luminal gastrointestinal tract.

115. The method according to any of claims 73 to 114, wherein the method is a method for treating a subject by creating an ostomy or fistula in the subject.

116. The method according to any of claims 73 to 115, wherein the method is a method for treating a subject by facilitating therapeutic interventional endoscopy.

117. The method according to any of claims 73 to 116, wherein the method is a method for treating a subject by facilitating placement of a lumen-apposing metal stent (LAMS) in a subject.

118. The method according to any of claims 73 to 117, wherein the method is a method for treating a subject by facilitating an endoscopic ultrasound guided gastroenterostomy (EUS-GE) procedure.

119. The method according to any of claims 73 to 118, wherein the method is a method for treating a subject by facilitating an endoscopic gastrojejunostomy procedure.

120. The method according to any of claims 112-119, wherein the subject is human.
